SEQUENCE LISTING (11 sequences)

SEQ ID NO: 1 - protein, 8 aa, unknown
  amino acid sequence of FLAG-tag
DYKDDDDK

SEQ ID NO: 2 - DNA, 5067 bp, artificial
  nucleic acid sequence of FLAG-TAT-HA Utrophin (delta)R7-22 construct
atggactacaaggacgacgatgacaagggctacggccgcaagaaacgccgccagcgccgccgcggtggatccaccatgtccggctatccatatgacgtcccagactatgctggctccatggccaagtatggggaccttgaagccaggcctgatgatgggcagaacgaattcagtgacatcattaagtccagatctgatgaacacaatgatgtacagaagaaaacctttaccaaatggataaacgctcgattttccaagagtgggaaaccacccatcagtgatatgttctcagacctcaaagatgggagaaagctcttggatcttctcgaaggcctcacaggaacatcattgccaaaggaacgtggttccacaagggtgcatgccttaaacaatgtcaaccgagtgctacaggttttacatcagaacaatgtggacttggtgaatattggaggcacggacattgtggatggaaatcccaagctgactttagggttactctggagcatcattctgcactggcaggtgaaggatgtcatgaaagatatcatgtcagacctgcagcagacaaacagcgagaagatcctgctgagctgggtgcggcagaccaccaggccctacagtcaagtcaacgtcctcaacttcaccaccagctggaccgatggactcgcgttcaacgccgtgctccaccggcacaaaccagatctcttcagctgggacagagtggtcaaaatgtccccaattgagagacttgaacatgcttttagcaaggcccacacttatttgggaattgaaaagcttctagatcctgaagatgttgctgtgcatctccctgacaagaaatccataattatgtatttaacgtctctgtttgaggtgcttcctcagcaagtcacgatagatgccatccgagaggtggagactctcccaaggaagtataagaaagaatgtgaagaggaagaaattcatatccagagtgcagtgctggcagaggaaggccagagtccccgagctgagacccctagcaccgtcactgaagtggacatggatttggacagctaccagatagcgctagaggaagtgctgacgtggctgctgtccgcggaggacacgttccaggagcaagatgacatttctgatgatgtcgaagaagtcaaagagcagtttgctacccatgaaacttttatgatggagctgacagcacaccagagcagcgtggggagcgtcctgcaggctggcaaccagctgatgacacaagggactctgtcagaggaggaggagtttgagatccaggaacagatgaccttgctgaatgcaaggtgggaggcgctccgggtggagagcatggagaggcagtcccggctgcacgacgctctgatggagctgcagaagaaacagctgcagcagctctcaagctggctggccctcacagaagagcgcattcagaagatggagagcctcccgctgggtgatgacctgccctccctgcagaagctgcttcaagaacataaaagtttgcaaaatgaccttgaagctgaacaggtgaaggtaaattccttaactcacatggtggtgattgtggatgaaaacagtggggagagtgccacagctcttctggaagatcagttacagaaactgggtgagcgctggacagctgtatgccgctggactgaagaacgttggaacaggttgcaagaaatcagtattctgtggcaggaattattggaagagcagtgtctgttggaggcttggctcaccgaaaaggaagaggctttgaataaagttcaaaccagcaactttaaagaccagaaggaactaagtgtcagtgtccggcgtctggctatattgaaggaagacatggaaatgaagaggcagactctggatcaactgagtgagattggccaggatgtgggccaattactcagtaatcccaaggcatctaagaagatgaacagtgactctgaggagctaacacagagatgggattctctggttcagagactcgaagactcttctaaccaggtgactcaggcggtagcgaagctcggcatgtcccagattccacagaaggacctattggagaccgttcatgtgagagaacaagggatggtgaagaagcccaagcaggaactgcctcctcctcccccaccaaagaagagacagattcacgtggacgtggaggccaagaaaaagtttgatgctataagtacagagctgctgaactggattttgaaatcaaagactgccattcagaacacagagatgaaagaatataagaagtcgcaggagacctcaggaatgaaaaagaaattgaagggattagagaaagaacagaaggaaaatctgccccgactggacgaactgaatcaaaccggacaaaccctccgggagcaaatgggaaaagaaggcctttccactgaagaagtaaacgatgttctggaaagggtttcgttggagtggaagatgatatctcagcagctagaagatctgggaaggaagatccagctgcaggaagatataaatgcttattttaagcagcttgatgccattgaggagaccatcaaggagaaggaagagtggctgaggggcacacccatttctgaatcgccccggcagcccttgccaggcttaaaggattcttgccagagggaactgacagatctccttggccttcaccccagaattgagacgctgtgtgcaagctgttcagccctgaagtctcagccctgtgtcccaggttttgtccagcagggttttgacgaccttcgacatcattaccaggctgtgcggaaggctttagaggaataccaacaacaactagaaaatgagctgaagagccagcctggacccgcgtatttggacacactgaataccctgaaaaaaatgctaagcgagtcagaaaaggcggcccaggcctctctgaatgccctgaacgatcccatagcggtggagcaggccctgcaggagaaaaaggcccttgatgaaacccttgagaatcagaaacatacgttacataagctttcagaagaaacgaagactttggagaaaaatatgcttcctgatgtggggaaaatgtataaacaagaatttgatgatgtccaaggcagatggaataaagtaaagaccaaggtttccagagacttacacttgctcgaggaaatcgcccacagagattttgggccatcttctcaacactttctgtccacttcagtccagctgccgtggcagagatccatttcacataataaagtgccctattacatcaaccatcaaacacagacaacctgttgggatcatcctaaaatgactgagctcttccaatcccttgctgatctgaataatgtacgtttctctgcctaccgcacagcaatcaaaattcgaaggctgcaaaaagcattatgtctggatctcttagagctgaatacgacgaatgaagttttcaagcagcacaaactgaaccaaaatgatcagctcctgagtgtcccagacgtcatcaactgtctgaccaccacttacgatgggcttgagcagctgcacaaggacttggtcaatgttccactctgcgtcgatatgtgtctcaactggctgctcaacgtatacgacacgggccggactggaaaaattcgggtacagagtctgaagattggattgatgtctctctccaaaggcctcttagaagagaaatacagatgtctctttaaggaggtggcagggccaacagagatgtgtgaccagcggcagcttggcctgctacttcacgatgccatccagatccctaggcagctgggggaagtagcagcctttgggggcagtaacattgagcccagtgtccgcagctgcttccagcagaataacaacaagccagaaatcagtgtgaaggagtttatagactggatgcatttggaaccccagtccatggtgtggttgccggttctgcatcgggtcgcagctgctgagactgcaaaacatcaggccaaatgcaacatctgcaaagaatgcccgattgttgggttcagatacaggagcctaaagcattttaattatgatgtctgccagagttgcttcttttctggaagaacagcaaagggccacaagttacattacccgatggtagaatactgcataccgacaacatctggggaagatgtgagagatttcactaaggtgctgaagaacaagttcaggtccaagaaatattttgccaaacatcctcggcttggctacctgcctgtccagaccgtgctggaaggggacaacttagaaactcctatcacgctcatcagtatgtggccagagcactatgacccctcccagtcccctcagctgtttcatgatgacacccactcaagaatagagcaatacgctacacgactggcccagatggaaaggacaaacgggtccttcctaactgatagcagctctacaacaggaagcgtggaggatgagcatgccctcatccagcagtactgccagaccctgggcggggagtcacctgtgagtcagccgcagagtccagctcagatcctgaagtccgtggagagggaagagcgtggggaactggagcggatcattgctgacttggaggaagagcaaagaaatctgcaggtggagtatgagcagctgaaggagcagcacctaagaaggggtctccctgtgggctcccctccagactccatcgtatctcctcaccacacatctgaggactcagaacttatagcagaagctaaactcctgcggcagcacaaagggcggctggaggcgaggatgcaaattttggaagatcacaataaacagctggagtctcagctgcaccgcctcagacagctcctggagcagcctgactctgactcccgcatcaatggtgtctccccctgggcttccccacagcattctgcattgagctactcacttgacactgacccaggcccacagttccaccaggcagcatctgaggacctgctggccccacctcacgacactagcacggacctcacggacgtgatggagcagatcaacagcacgtttccctcttgcagctcaaatgtccccagcaggccacaggcaatgtga

SEQ ID NO: 3 - protein, 1688 aa, artificial
  amino acid sequence of FLAG-TAT-HA Utrophin (delta)R7-22 construct
MDYKDDDDKGYGRKKRRQRRRGGSTMSGYPYDVPDYAGSMAKYGDLEARPDDGQNEFSDIIKSRSDEHNDVQKKTFTKWINARFSKSGKPPISDMFSDLKDGRKLLDLLEGLTGTSLPKERGSTRVHALNNVNRVLQVLHQNNVDLVNIGGTDIVDGNPKLTLGLLWSIILHWQVKDVMKDIMSDLQQTNSEKILLSWVRQTTRPYSQVNVLNFTTSWTDGLAFNAVLHRHKPDLFSWDRVVKMSPIERLEHAFSKAHTYLGIEKLLDPEDVAVHLPDKKSIIMYLTSLFEVLPQQVTIDAIREVETLPRKYKKECEEEEIHIQSAVLAEEGQSPRAETPSTVTEVDMDLDSYQIALEEVLTWLLSAEDTFQEQDDISDDVEEVKEQFATHETFMMELTAHQSSVGSVLQAGNQLMTQGTLSEEEEFEIQEQMTLLNARWEALRVESMERQSRLHDALMELQKKQLQQLSSWLALTEERIQKMESLPLGDDLPSLQKLLQEHKSLQNDLEAEQVKVNSLTHMVVIVDENSGESATALLEDQLQKLGERWTAVCRWTEERWNRLQEISILWQELLEEQCLLEAWLTEKEEALNKVQTSNFKDQKELSVSVRRLAILKEDMEMKRQTLDQLSEIGQDVGQLLSNPKASKKMNSDSEELTQRWDSLVQRLEDSSNQVTQAVAKLGMSQIPQKDLLETVHVREQGMVKKPKQELPPPPPPKKRQIHVDVEAKKKFDAISTELLNWILKSKTAIQNTEMKEYKKSQETSGMKKKLKGLEKEQKENLPRLDELNQTGQTLREQMGKEGLSTEEVNDVLERVSLEWKMISQQLEDLGRKIQLQEDINAYFKQLDAIEETIKEKEEWLRGTPISESPRQPLPGLKDSCQRELTDLLGLHPRIETLCASCSALKSQPCVPGFVQQGFDDLRHHYQAVRKALEEYQQQLENELKSQPGPAYLDTLNTLKKMLSESEKAAQASLNALNDPIAVEQALQEKKALDETLENQKHTLHKLSEETKTLEKNMLPDVGKMYKQEFDDVQGRWNKVKTKVSRDLHLLEEIAHRDFGPSSQHFLSTSVQLPWQRSISHNKVPYYINHQTQTTCWDHPKMTELFQSLADLNNVRFSAYRTAIKIRRLQKALCLDLLELNTTNEVFKQHKLNQNDQLLSVPDVINCLTTTYDGLEQLHKDLVNVPLCVDMCLNWLLNVYDTGRTGKIRVQSLKIGLMSLSKGLLEEKYRCLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKEQHLRRGLPVGSPPDSIVSPHHTSEDSELIAEAKLLRQHKGRLEARMQILEDHNKQLESQLHRLRQLLEQPDSDSRINGVSPWASPQHSALSYSLDTDPGPQFHQAASEDLLAPPHDTSTDLTDVMEQINSTFPSCSSNVPSRPQAM

SEQ ID NO: 4 - DNA, 6324 bp, artificial
  nucleic acid sequence of FLAG-TAT-HA Utrophin (delta)R11-22 construct
atggactacaaggacgacgatgacaagggctacggccgcaagaaacgccgccagcgccgccgcggtggatccaccatgtccggctatccatatgacgtcccagactatgctggctccatggccaagtatggggaccttgaagccaggcctgatgatgggcagaacgaattcagtgacatcattaagtccagatctgatgaacacaatgatgtacagaagaaaacctttaccaaatggataaacgctcgattttccaagagtgggaaaccacccatcagtgatatgttctcagacctcaaagatgggagaaagctcttggatcttctcgaaggcctcacaggaacatcattgccaaaggaacgtggttccacaagggtgcatgccttaaacaatgtcaaccgagtgctacaggttttacatcagaacaatgtggacttggtgaatattggaggcacggacattgtggatggaaatcccaagctgactttagggttactctggagcatcattctgcactggcaggtgaaggatgtcatgaaagatatcatgtcagacctgcagcagacaaacagcgagaagatcctgctgagctgggtgcggcagaccaccaggccctacagtcaagtcaacgtcctcaacttcaccaccagctggaccgatggactcgcgttcaacgccgtgctccaccggcacaaaccagatctcttcagctgggacagagtggtcaaaatgtccccaattgagagacttgaacatgcttttagcaaggcccacacttatttgggaattgaaaagcttctagatcctgaagatgttgctgtgcatctccctgacaagaaatccataattatgtatttaacgtctctgtttgaggtgcttcctcagcaagtcacgatagatgccatccgagaggtggagactctcccaaggaagtataagaaagaatgtgaagaggaagaaattcatatccagagtgcagtgctggcagaggaaggccagagtccccgagctgagacccctagcaccgtcactgaagtggacatggatttggacagctaccagatagcgctagaggaagtgctgacgtggctgctgtccgcggaggacacgttccaggagcaagatgacatttctgatgatgtcgaagaagtcaaagagcagtttgctacccatgaaacttttatgatggagctgacagcacaccagagcagcgtggggagcgtcctgcaggctggcaaccagctgatgacacaagggactctgtcagaggaggaggagtttgagatccaggaacagatgaccttgctgaatgcaaggtgggaggcgctccgggtggagagcatggagaggcagtcccggctgcacgacgctctgatggagctgcagaagaaacagctgcagcagctctcaagctggctggccctcacagaagagcgcattcagaagatggagagcctcccgctgggtgatgacctgccctccctgcagaagctgcttcaagaacataaaagtttgcaaaatgaccttgaagctgaacaggtgaaggtaaattccttaactcacatggtggtgattgtggatgaaaacagtggggagagtgccacagctcttctggaagatcagttacagaaactgggtgagcgctggacagctgtatgccgctggactgaagaacgttggaacaggttgcaagaaatcagtattctgtggcaggaattattggaagagcagtgtctgttggaggcttggctcaccgaaaaggaagaggctttgaataaagttcaaaccagcaactttaaagaccagaaggaactaagtgtcagtgtccggcgtctggctatattgaaggaagacatggaaatgaagaggcagactctggatcaactgagtgagattggccaggatgtgggccaattactcagtaatcccaaggcatctaagaagatgaacagtgactctgaggagctaacacagagatgggattctctggttcagagactcgaagactcttctaaccaggtgactcaggcggtagcgaagctcggcatgtcccagattccacagaaggacctattggagaccgttcatgtgagagaacaagggatggtgaagaagcccaagcaggaactgcctcctcctcccccaccaaagaagagacagattcacgtggacgtggaggccaagaaaaagtttgatgctataagtacagagctgctgaactggattttgaaatcaaagactgccattcagaacacagagatgaaagaatataagaagtcgcaggagacctcaggaatgaaaaagaaattgaagggattagagaaagaacagaaggaaaatctgccccgactggacgaactgaatcaaaccggacaaaccctccgggagcaaatgggaaaagaaggcctttccactgaagaagtaaacgatgttctggaaagggtttcgttggagtggaagatgatatctcagcagctagaagatctgggaaggaagatccagctgcaggaagatataaatgcttattttaagcagcttgatgccattgaggagaccatcaaggagaaggaagagtggctgaggggcacacccatttctgaatcgccccggcagcccttgccaggcttaaaggattcttgccagagggaactgacagatctccttggccttcaccccagaattgagacgctgtgtgcaagctgttcagccctgaagtctcagccctgtgtcccaggttttgtccagcagggttttgacgaccttcgacatcattaccaggctgtgcggaaggctttagaggaataccaacaacaactagaaaatgagctgaagagccagcctggacccgcgtatttggacacactgaataccctgaaaaaaatgctaagcgagtcagaaaaggcggcccaggcctctctgaatgccctgaacgatcccatagcggtggagcaggccctgcaggagaaaaaggcccttgatgaaacccttgagaatcagaaacatacgttacataagctttcagaagaaacgaagactttggagaaaaatatgcttcctgatgtggggaaaatgtataaacaagaatttgatgatgtccaaggcagatggaataaagtaaagaccaaggtttccagagacttacacttgctcgaggaaatcacccccagactccgagattttgaggctgattcagaagtcattgagaagtgggtgagtggcatcaaagacttcctcatgaaagaacaggctgctcaaggagacgctgctgcgctgcagagccagcttgaccaatgtgctacgtttgctaatgaaatcgaaaccatcgagtcatctctgaagaacatgagggaagtagagactagccttcagaggtgtccagtcactggagtcaagacatgggtacaggcaagactagtggattaccaatcccaactggagaaattcagcaaagagattgctattcaaaaaagcaggctgtcagatagtcaagaaaaagccctgaacttgaaaaaggatttggctgagatgcaggagtggatggcacaggctgaagaggactacctggagagggacttcgagtacaaatctccagaagaactcgagagtgcggtggaggaaatgaagagggcaaaagaggaggtgctgcagaaggaggtgagggtgaaaattctgaaggacagcatcaagctggtggctgccaaggtgccctctggtggccaggagttgacgtcggaattcaacgaggtgctggagagctaccagcttctgtgcaatagaattcgagggaagtgccacacactggaggaggtctggtcttgctgggtggagctgcttcactatctggacctggagaccacgtggttgaacaccttggaggagcgcgtgaggagcacggaggccctgcctgagagggcagaagctgttcatgaagctctggagtctcttgagtctgttttgcgccatccggcggataatcgcacccagattcgggaacttgggcagactctgattgatggtggaatcctggatgacataatcagcgagaagctggaggcttttaacagccgctacgaagagctgagtcacttggcggagagcaaacagatttctttggagaagcaactccaggtcctccgcgaaactgaccacatgcttcaggtgctgaaggagagcctgggggagctggacaaacagcttaccacatacctgacggacaggatcgatgccttccaactgccacaggaagctcagaagatccaagccgaaatctcagcccatgagctcaccctggaggagctgaggaagaatgtgcgctcccagcccccgacgtcccctgagggcagggccaccagaggaggaagtcagatggacatgctacagaggaaacttcgagaggtctccaccaaattccagcttgcccacagagattttgggccatcttctcaacactttctgtccacttcagtccagctgccgtggcagagatccatttcacataataaagtgccctattacatcaaccatcaaacacagacaacctgttgggatcatcctaaaatgactgagctcttccaatcccttgctgatctgaataatgtacgtttctctgcctaccgcacagcaatcaaaattcgaaggctgcaaaaagcattatgtctggatctcttagagctgaatacgacgaatgaagttttcaagcagcacaaactgaaccaaaatgatcagctcctgagtgtcccagacgtcatcaactgtctgaccaccacttacgatgggcttgagcagctgcacaaggacttggtcaatgttccactctgcgtcgatatgtgtctcaactggctgctcaacgtatacgacacgggccggactggaaaaattcgggtacagagtctgaagattggattgatgtctctctccaaaggcctcttagaagagaaatacagatgtctctttaaggaggtggcagggccaacagagatgtgtgaccagcggcagcttggcctgctacttcacgatgccatccagatccctaggcagctgggggaagtagcagcctttgggggcagtaacattgagcccagtgtccgcagctgcttccagcagaataacaacaagccagaaatcagtgtgaaggagtttatagactggatgcatttggaaccccagtccatggtgtggttgccggttctgcatcgggtcgcagctgctgagactgcaaaacatcaggccaaatgcaacatctgcaaagaatgcccgattgttgggttcagatacaggagcctaaagcattttaattatgatgtctgccagagttgcttcttttctggaagaacagcaaagggccacaagttacattacccgatggtagaatactgcataccgacaacatctggggaagatgtgagagatttcactaaggtgctgaagaacaagttcaggtccaagaaatattttgccaaacatcctcggcttggctacctgcctgtccagaccgtgctggaaggggacaacttagaaactcctatcacgctcatcagtatgtggccagagcactatgacccctcccagtcccctcagctgtttcatgatgacacccactcaagaatagagcaatacgctacacgactggcccagatggaaaggacaaacgggtccttcctaactgatagcagctctacaacaggaagcgtggaggatgagcatgccctcatccagcagtactgccagaccctgggcggggagtcacctgtgagtcagccgcagagtccagctcagatcctgaagtccgtggagagggaagagcgtggggaactggagcggatcattgctgacttggaggaagagcaaagaaatctgcaggtggagtatgagcagctgaaggagcagcacctaagaaggggtctccctgtgggctcccctccagactccatcgtatctcctcaccacacatctgaggactcagaacttatagcagaagctaaactcctgcggcagcacaaagggcggctggaggcgaggatgcaaattttggaagatcacaataaacagctggagtctcagctgcaccgcctcagacagctcctggagcagcctgactctgactcccgcatcaatggtgtctccccctgggcttccccacagcattctgcattgagctactcacttgacactgacccaggcccacagttccaccaggcagcatctgaggacctgctggccccacctcacgacactagcacggacctcacggacgtgatggagcagatcaacagcacgtttccctcttgcagctcaaatgtccccagcaggccacaggcaatgtga

SEQ ID NO: 5 - protein, 2107 aa, artificial
  amino acid sequence of FLAG-TAT-HA Utrophin (delta)R11-22 construct
MDYKDDDDKGYGRKKRRQRRRGGSTMSGYPYDVPDYAGSMAKYGDLEARPDDGQNEFSDIIKSRSDEHNDVQKKTFTKWINARFSKSGKPPISDMFSDLKDGRKLLDLLEGLTGTSLPKERGSTRVHALNNVNRVLQVLHQNNVDLVNIGGTDIVDGNPKLTLGLLWSIILHWQVKDVMKDIMSDLQQTNSEKILLSWVRQTTRPYSQVNVLNFTTSWTDGLAFNAVLHRHKPDLFSWDRVVKMSPIERLEHAFSKAHTYLGIEKLLDPEDVAVHLPDKKSIIMYLTSLFEVLPQQVTIDAIREVETLPRKYKKECEEEEIHIQSAVLAEEGQSPRAETPSTVTEVDMDLDSYQIALEEVLTWLLSAEDTFQEQDDISDDVEEVKEQFATHETFMMELTAHQSSVGSVLQAGNQLMTQGTLSEEEEFEIQEQMTLLNARWEALRVESMERQSRLHDALMELQKKQLQQLSSWLALTEERIQKMESLPLGDDLPSLQKLLQEHKSLQNDLEAEQVKVNSLTHMVVIVDENSGESATALLEDQLQKLGERWTAVCRWTEERWNRLQEISILWQELLEEQCLLEAWLTEKEEALNKVQTSNFKDQKELSVSVRRLAILKEDMEMKRQTLDQLSEIGQDVGQLLSNPKASKKMNSDSEELTQRWDSLVQRLEDSSNQVTQAVAKLGMSQIPQKDLLETVHVREQGMVKKPKQELPPPPPPKKRQIHVDVEAKKKFDAISTELLNWILKSKTAIQNTEMKEYKKSQETSGMKKKLKGLEKEQKENLPRLDELNQTGQTLREQMGKEGLSTEEVNDVLERVSLEWKMISQQLEDLGRKIQLQEDINAYFKQLDAIEETIKEKEEWLRGTPISESPRQPLPGLKDSCQRELTDLLGLHPRIETLCASCSALKSQPCVPGFVQQGFDDLRHHYQAVRKALEEYQQQLENELKSQPGPAYLDTLNTLKKMLSESEKAAQASLNALNDPIAVEQALQEKKALDETLENQKHTLHKLSEETKTLEKNMLPDVGKMYKQEFDDVQGRWNKVKTKVSRDLHLLEEITPRLRDFEADSEVIEKWVSGIKDFLMKEQAAQGDAAALQSQLDQCATFANEIETIESSLKNMREVETSLQRCPVTGVKTWVQARLVDYQSQLEKFSKEIAIQKSRLSDSQEKALNLKKDLAEMQEWMAQAEEDYLERDFEYKSPEELESAVEEMKRAKEEVLQKEVRVKILKDSIKLVAAKVPSGGQELTSEFNEVLESYQLLCNRIRGKCHTLEEVWSCWVELLHYLDLETTWLNTLEERVRSTEALPERAEAVHEALESLESVLRHPADNRTQIRELGQTLIDGGILDDIISEKLEAFNSRYEELSHLAESKQISLEKQLQVLRETDHMLQVLKESLGELDKQLTTYLTDRIDAFQLPQEAQKIQAEISAHELTLEELRKNVRSQPPTSPEGRATRGGSQMDMLQRKLREVSTKFQLAHRDFGPSSQHFLSTSVQLPWQRSISHNKVPYYINHQTQTTCWDHPKMTELFQSLADLNNVRFSAYRTAIKIRRLQKALCLDLLELNTTNEVFKQHKLNQNDQLLSVPDVINCLTTTYDGLEQLHKDLVNVPLCVDMCLNWLLNVYDTGRTGKIRVQSLKIGLMSLSKGLLEEKYRCLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKEQHLRRGLPVGSPPDSIVSPHHTSEDSELIAEAKLLRQHKGRLEARMQILEDHNKQLESQLHRLRQLLEQPDSDSRINGVSPWASPQHSALSYSLDTDPGPQFHQAASEDLLAPPHDTSTDLTDVMEQINSTFPSCSSNVPSRPQAM

SEQ ID NO: 6 - DNA, 4380 bp, artificial
  nucleic acid sequence of TAT-HA Utrophin (delta)R4-21 construct
atgggctacggccgcaagaaacgccgccagcgccgccgcggtggatccaccatgtccggctatccatatgacgtcccagactatgctggctccatggccaagtatggggaccttgaagccaggcctgatgatgggcagaacgaattcagtgacatcattaagtccagatctgatgaacacaatgatgtacagaagaaaacctttaccaaatggataaacgctcgattttccaagagtgggaaaccacccatcagtgatatgttctcagacctcaaagatgggagaaagctcttggatcttctcgaaggcctcacaggaacatcattgccaaaggaacgtggttccacaagggtgcatgccttaaacaatgtcaaccgagtgctacaggttttacatcagaacaatgtggacttggtgaatattggaggcacggacattgtggatggaaatcccaagctgactttagggttactctggagcatcattctgcactggcaggtgaaggatgtcatgaaagatatcatgtcagacctgcagcagacaaacagcgagaagatcctgctgagctgggtgcggcagaccaccaggccctacagtcaagtcaacgtcctcaacttcaccaccagctggaccgatggactcgcgttcaacgccgtgctccaccggcacaaaccagatctcttcagctgggacagagtggtcaaaatgtccccaattgagagacttgaacatgcttttagcaaggcccacacttatttgggaattgaaaagcttctagatcctgaagatgttgctgtgcatctccctgacaagaaatccataattatgtatttaacgtctctgtttgaggtgcttcctcagcaagtcacgatagatgccatccgagaggtggagactctcccaaggaagtataagaaagaatgtgaagaggaagaaattcatatccagagtgcagtgctggcagaggaaggccagagtccccgagctgagacccctagcaccgtcactgaagtggacatggatttggacagctaccagatagcgctagaggaagtgctgacgtggctgctgtccgcggaggacacgttccaggagcaagatgacatttctgatgatgtcgaagaagtcaaagagcagtttgctacccatgaaacttttatgatggagctgacagcacaccagagcagcgtggggagcgtcctgcaggctggcaaccagctgatgacacaagggactctgtcagaggaggaggagtttgagatccaggaacagatgaccttgctgaatgcaaggtgggaggcgctccgggtggagagcatggagaggcagtcccggctgcacgacgctctgatggagctgcagaagaaacagctgcagcagctctcaagctggctggccctcacagaagagcgcattcagaagatggagagcctcccgctgggtgatgacctgccctccctgcagaagctgcttcaagaacataaaagtttgcaaaatgaccttgaagctgaacaggtgaaggtaaattccttaactcacatggtggtgattgtggatgaaaacagtggggagagtgccacagctcttctggaagatcagttacagaaactgggtgagcgctggacagctgtatgccgctggactgaagaacgttggaacaggttgcaagaaatcagtattctgtggcaggaattattggaagagcagtgtctgttggaggcttggctcaccgaaaaggaagaggctttgaataaagttcaaaccagcaactttaaagaccagaaggaactaagtgtcagtgtccggcgtctggctatattgaaggaagacatggaaatgaagaggcagactctggatcaactgagtgagattggccaggatgtgggccaattactcagtaatcccaaggcatctaagaagatgaacagtgactctgaggagctaacacagagatgggattctctggttcagagactcgaagactcttctaaccaggtgactcaggcggtagcgaagctcggcatgtcccagattccacagaaggacctattggagaccgttcatgtgagagaacaagggatggtgaagaagcccaagcaggaactgcctcctcctcccccaccaaagaagagacagattcacgtggacttagagaaactccgagacctgcagggagctatggacgacctggacgcagacatgaaggaggtggaggctgtgcggaatggctggaagcccgtgggagacctgcttatagactccctgcaggatcacatcgagaaaaccctggcgtttagagaagaaattgcaccaatcaacttaaaagtaaaaacaatgaatgacctgtccagtcagctgtctccacttgacttgcatccatctctaaagatgtctcgccagctggatgaccttaatatgcgatggaaacttctacaggtttccgtggacgatcgccttaagcagctccaggaagcccacagagattttgggccatcttctcaacactttctgtccacttcagtccagctgccgtggcagagatccatttcacataataaagtgccctattacatcaaccatcaaacacagacaacctgttgggatcatcctaaaatgactgagctcttccaatcccttgctgatctgaataatgtacgtttctctgcctaccgcacagcaatcaaaattcgaaggctgcaaaaagcattatgtctggatctcttagagctgaatacgacgaatgaagttttcaagcagcacaaactgaaccaaaatgatcagctcctgagtgtcccagacgtcatcaactgtctgaccaccacttacgatgggcttgagcagctgcacaaggacttggtcaatgttccactctgcgtcgatatgtgtctcaactggctgctcaacgtatacgacacgggccggactggaaaaattcgggtacagagtctgaagattggattgatgtctctctccaaaggcctcttagaagagaaatacagatgtctctttaaggaggtggcagggccaacagagatgtgtgaccagcggcagcttggcctgctacttcacgatgccatccagatccctaggcagctgggggaagtagcagcctttgggggcagtaacattgagcccagtgtccgcagctgcttccagcagaataacaacaagccagaaatcagtgtgaaggagtttatagactggatgcatttggaaccccagtccatggtgtggttgccggttctgcatcgggtcgcagctgctgagactgcaaaacatcaggccaaatgcaacatctgcaaagaatgcccgattgttgggttcagatacaggagcctaaagcattttaattatgatgtctgccagagttgcttcttttctggaagaacagcaaagggccacaagttacattacccgatggtagaatactgcataccgacaacatctggggaagatgtgagagatttcactaaggtgctgaagaacaagttcaggtccaagaaatattttgccaaacatcctcggcttggctacctgcctgtccagaccgtgctggaaggggacaacttagaaactcctatcacgctcatcagtatgtggccagagcactatgacccctcccagtcccctcagctgtttcatgatgacacccactcaagaatagagcaatacgctacacgactggcccagatggaaaggacaaacgggtccttcctaactgatagcagctctacaacaggaagcgtggaggatgagcatgccctcatccagcagtactgccagaccctgggcggggagtcacctgtgagtcagccgcagagtccagctcagatcctgaagtccgtggagagggaagagcgtggggaactggagcggatcattgctgacttggaggaagagcaaagaaatctgcaggtggagtatgagcagctgaaggagcagcacctaagaaggggtctccctgtgggctcccctccagactccatcgtatctcctcaccacacatctgaggactcagaacttatagcagaagctaaactcctgcggcagcacaaagggcggctggaggcgaggatgcaaattttggaagatcacaataaacagctggagtctcagctgcaccgcctcagacagctcctggagcagcctgactctgactcccgcatcaatggtgtctccccctgggcttccccacagcattctgcattgagctactcacttgacactgacccaggcccacagttccaccaggcagcatctgaggacctgctggccccacctcacgacactagcacggacctcacggacgtgatggagcagatcaacagcacgtttccctcttgcagctcaaatgtccccagcaggccacaggcaatgtga

SEQ ID NO: 7 - protein, 1459 aa, artificial
  amino acid sequence of TAT-HA Utrophin (delta)R4-21 construct
MGYGRKKRRQRRRGGSTMSGYPYDVPDYAGSMAKYGDLEARPDDGQNEFSDIIKSRSDEHNDVQKKTFTKWINARFSKSGKPPISDMFSDLKDGRKLLDLLEGLTGTSLPKERGSTRVHALNNVNRVLQVLHQNNVDLVNIGGTDIVDGNPKLTLGLLWSIILHWQVKDVMKDIMSDLQQTNSEKILLSWVRQTTRPYSQVNVLNFTTSWTDGLAFNAVLHRHKPDLFSWDRVVKMSPIERLEHAFSKAHTYLGIEKLLDPEDVAVHLPDKKSIIMYLTSLFEVLPQQVTIDAIREVETLPRKYKKECEEEEIHIQSAVLAEEGQSPRAETPSTVTEVDMDLDSYQIALEEVLTWLLSAEDTFQEQDDISDDVEEVKEQFATHETFMMELTAHQSSVGSVLQAGNQLMTQGTLSEEEEFEIQEQMTLLNARWEALRVESMERQSRLHDALMELQKKQLQQLSSWLALTEERIQKMESLPLGDDLPSLQKLLQEHKSLQNDLEAEQVKVNSLTHMVVIVDENSGESATALLEDQLQKLGERWTAVCRWTEERWNRLQEISILWQELLEEQCLLEAWLTEKEEALNKVQTSNFKDQKELSVSVRRLAILKEDMEMKRQTLDQLSEIGQDVGQLLSNPKASKKMNSDSEELTQRWDSLVQRLEDSSNQVTQAVAKLGMSQIPQKDLLETVHVREQGMVKKPKQELPPPPPPKKRQIHVDLEKLRDLQGAMDDLDADMKEVEAVRNGWKPVGDLLIDSLQDHIEKTLAFREEIAPINLKVKTMNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELNTTNEVFKQHKLNQNDQLLSVPDVINCLTTTYDGLEQLHKDLVNVPLCVDMCLNWLLNVYDTGRTGKIRVQSLKIGLMSLSKGLLEEKYRCLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKEQHLRRGLPVGSPPDSIVSPHHTSEDSELIAEAKLLRQHKGRLEARMQILEDHNKQLESQLHRLRQLLEQPDSDSRINGVSPWASPQHSALSYSLDTDPGPQFHQAASEDLLAPPHDTSTDLTDVMEQINSTFPSCSSNVPSRPQAM

SEQ ID NO: 8 - DNA, 4323 bp, artificial
  nucleic acid sequence of TAT Utrophin (delta)R4-21 construct
atgggctacggccgcaagaaacgccgccagcgccgccgcgccaagtatggggaccttgaagccaggcctgatgatgggcagaacgaattcagtgacatcattaagtccagatctgatgaacacaatgatgtacagaagaaaacctttaccaaatggataaacgctcgattttccaagagtgggaaaccacccatcagtgatatgttctcagacctcaaagatgggagaaagctcttggatcttctcgaaggcctcacaggaacatcattgccaaaggaacgtggttccacaagggtgcatgccttaaacaatgtcaaccgagtgctacaggttttacatcagaacaatgtggacttggtgaatattggaggcacggacattgtggatggaaatcccaagctgactttagggttactctggagcatcattctgcactggcaggtgaaggatgtcatgaaagatatcatgtcagacctgcagcagacaaacagcgagaagatcctgctgagctgggtgcggcagaccaccaggccctacagtcaagtcaacgtcctcaacttcaccaccagctggaccgatggactcgcgttcaacgccgtgctccaccggcacaaaccagatctcttcagctgggacagagtggtcaaaatgtccccaattgagagacttgaacatgcttttagcaaggcccacacttatttgggaattgaaaagcttctagatcctgaagatgttgctgtgcatctccctgacaagaaatccataattatgtatttaacgtctctgtttgaggtgcttcctcagcaagtcacgatagatgccatccgagaggtggagactctcccaaggaagtataagaaagaatgtgaagaggaagaaattcatatccagagtgcagtgctggcagaggaaggccagagtccccgagctgagacccctagcaccgtcactgaagtggacatggatttggacagctaccagatagcgctagaggaagtgctgacgtggctgctgtccgcggaggacacgttccaggagcaagatgacatttctgatgatgtcgaagaagtcaaagagcagtttgctacccatgaaacttttatgatggagctgacagcacaccagagcagcgtggggagcgtcctgcaggctggcaaccagctgatgacacaagggactctgtcagaggaggaggagtttgagatccaggaacagatgaccttgctgaatgcaaggtgggaggcgctccgggtggagagcatggagaggcagtcccggctgcacgacgctctgatggagctgcagaagaaacagctgcagcagctctcaagctggctggccctcacagaagagcgcattcagaagatggagagcctcccgctgggtgatgacctgccctccctgcagaagctgcttcaagaacataaaagtttgcaaaatgaccttgaagctgaacaggtgaaggtaaattccttaactcacatggtggtgattgtggatgaaaacagtggggagagtgccacagctcttctggaagatcagttacagaaactgggtgagcgctggacagctgtatgccgctggactgaagaacgttggaacaggttgcaagaaatcagtattctgtggcaggaattattggaagagcagtgtctgttggaggcttggctcaccgaaaaggaagaggctttgaataaagttcaaaccagcaactttaaagaccagaaggaactaagtgtcagtgtccggcgtctggctatattgaaggaagacatggaaatgaagaggcagactctggatcaactgagtgagattggccaggatgtgggccaattactcagtaatcccaaggcatctaagaagatgaacagtgactctgaggagctaacacagagatgggattctctggttcagagactcgaagactcttctaaccaggtgactcaggcggtagcgaagctcggcatgtcccagattccacagaaggacctattggagaccgttcatgtgagagaacaagggatggtgaagaagcccaagcaggaactgcctcctcctcccccaccaaagaagagacagattcacgtggacttagagaaactccgagacctgcagggagctatggacgacctggacgcagacatgaaggaggtggaggctgtgcggaatggctggaagcccgtgggagacctgcttatagactccctgcaggatcacatcgagaaaaccctggcgtttagagaagaaattgcaccaatcaacttaaaagtaaaaacaatgaatgacctgtccagtcagctgtctccacttgacttgcatccatctctaaagatgtctcgccagctggatgaccttaatatgcgatggaaacttctacaggtttccgtggacgatcgccttaagcagctccaggaagcccacagagattttgggccatcttctcaacactttctgtccacttcagtccagctgccgtggcagagatccatttcacataataaagtgccctattacatcaaccatcaaacacagacaacctgttgggatcatcctaaaatgactgagctcttccaatcccttgctgatctgaataatgtacgtttctctgcctaccgcacagcaatcaaaattcgaaggctgcaaaaagcattatgtctggatctcttagagctgaatacgacgaatgaagttttcaagcagcacaaactgaaccaaaatgatcagctcctgagtgtcccagacgtcatcaactgtctgaccaccacttacgatgggcttgagcagctgcacaaggacttggtcaatgttccactctgcgtcgatatgtgtctcaactggctgctcaacgtatacgacacgggccggactggaaaaattcgggtacagagtctgaagattggattgatgtctctctccaaaggcctcttagaagagaaatacagatgtctctttaaggaggtggcagggccaacagagatgtgtgaccagcggcagcttggcctgctacttcacgatgccatccagatccctaggcagctgggggaagtagcagcctttgggggcagtaacattgagcccagtgtccgcagctgcttccagcagaataacaacaagccagaaatcagtgtgaaggagtttatagactggatgcatttggaaccccagtccatggtgtggttgccggttctgcatcgggtcgcagctgctgagactgcaaaacatcaggccaaatgcaacatctgcaaagaatgcccgattgttgggttcagatacaggagcctaaagcattttaattatgatgtctgccagagttgcttcttttctggaagaacagcaaagggccacaagttacattacccgatggtagaatactgcataccgacaacatctggggaagatgtgagagatttcactaaggtgctgaagaacaagttcaggtccaagaaatattttgccaaacatcctcggcttggctacctgcctgtccagaccgtgctggaaggggacaacttagaaactcctatcacgctcatcagtatgtggccagagcactatgacccctcccagtcccctcagctgtttcatgatgacacccactcaagaatagagcaatacgctacacgactggcccagatggaaaggacaaacgggtccttcctaactgatagcagctctacaacaggaagcgtggaggatgagcatgccctcatccagcagtactgccagaccctgggcggggagtcacctgtgagtcagccgcagagtccagctcagatcctgaagtccgtggagagggaagagcgtggggaactggagcggatcattgctgacttggaggaagagcaaagaaatctgcaggtggagtatgagcagctgaaggagcagcacctaagaaggggtctccctgtgggctcccctccagactccatcgtatctcctcaccacacatctgaggactcagaacttatagcagaagctaaactcctgcggcagcacaaagggcggctggaggcgaggatgcaaattttggaagatcacaataaacagctggagtctcagctgcaccgcctcagacagctcctggagcagcctgactctgactcccgcatcaatggtgtctccccctgggcttccccacagcattctgcattgagctactcacttgacactgacccaggcccacagttccaccaggcagcatctgaggacctgctggccccacctcacgacactagcacggacctcacggacgtgatggagcagatcaacagcacgtttccctcttgcagctcaaatgtccccagcaggccacaggcaatgtga

SEQ ID NO: 9 - protein, 1440 aa, artificial
  amino acid sequence of TAT Utrophin (delta)R4-21 construct
MGYGRKKRRQRRRAKYGDLEARPDDGQNEFSDIIKSRSDEHNDVQKKTFTKWINARFSKSGKPPISDMFSDLKDGRKLLDLLEGLTGTSLPKERGSTRVHALNNVNRVLQVLHQNNVDLVNIGGTDIVDGNPKLTLGLLWSIILHWQVKDVMKDIMSDLQQTNSEKILLSWVRQTTRPYSQVNVLNFTTSWTDGLAFNAVLHRHKPDLFSWDRVVKMSPIERLEHAFSKAHTYLGIEKLLDPEDVAVHLPDKKSIIMYLTSLFEVLPQQVTIDAIREVETLPRKYKKECEEEEIHIQSAVLAEEGQSPRAETPSTVTEVDMDLDSYQIALEEVLTWLLSAEDTFQEQDDISDDVEEVKEQFATHETFMMELTAHQSSVGSVLQAGNQLMTQGTLSEEEEFEIQEQMTLLNARWEALRVESMERQSRLHDALMELQKKQLQQLSSWLALTEERIQKMESLPLGDDLPSLQKLLQEHKSLQNDLEAEQVKVNSLTHMVVIVDENSGESATALLEDQLQKLGERWTAVCRWTEERWNRLQEISILWQELLEEQCLLEAWLTEKEEALNKVQTSNFKDQKELSVSVRRLAILKEDMEMKRQTLDQLSEIGQDVGQLLSNPKASKKMNSDSEELTQRWDSLVQRLEDSSNQVTQAVAKLGMSQIPQKDLLETVHVREQGMVKKPKQELPPPPPPKKRQIHVDLEKLRDLQGAMDDLDADMKEVEAVRNGWKPVGDLLIDSLQDHIEKTLAFREEIAPINLKVKTMNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELNTTNEVFKQHKLNQNDQLLSVPDVINCLTTTYDGLEQLHKDLVNVPLCVDMCLNWLLNVYDTGRTGKIRVQSLKIGLMSLSKGLLEEKYRCLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKEQHLRRGLPVGSPPDSIVSPHHTSEDSELIAEAKLLRQHKGRLEARMQILEDHNKQLESQLHRLRQLLEQPDSDSRINGVSPWASPQHSALSYSLDTDPGPQFHQAASEDLLAPPHDTSTDLTDVMEQINSTFPSCSSNVPSRPQAM

SEQ ID NO: 10 - protein, 1459 aa, artificial
  exemplary amino acid sequence including both TAT and HA peptide sequences
MYGRKKRRQRRRGGSTMSGYPYDVPDYAGSMAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWLTLTEERIQKMETCPLDDDVKSLQKLLEEHKSLQSDLEAEQVKVNSLTHMVVIVDENSGESATAILEDQLQKLGERWTAVCRWTEERWNRLQEINILWQELLEEQCLLKAWLTEKEEALNKVQTSNFKDQKELSVSVRRLAILKEDMEMKRQTLDQLSEIGQDVGQLLDNSKASKKINSDSEELTQRWDSLVQRLEDSSNQVTQAVAKLGMSQIPQKDLLETVRVREQAITKKSKQELPPPPPPKKRQIHVDLEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPITLISMWPEHYDPSQSPQLFHDDTHSRIEQYATRLAQMERTNGSFLTDSSSTTGSVEDEHALIQQYCQTLGGESPVSQPQSPAQILKSVEREERGELERIIADLEEEQRNLQVEYEQLKDQHLRRGLPVGSPPESIISPHHTSEDSELIAEAKLLRQHKGRLEARMQILEDHNKQLESQLHRLRQLLEQPESDSRINGVSPWASPQHSALSYSLDPDASGPQFHQAAGEDLLAPPHDTSTDLTEVMEQIHSTFPSCCPNVPSRPQAM

SEQ ID NO: 11 - protein, 1459 aa, artificial; identical to SEQ ID NO: 10
  exemplary amino acid sequence including a TAT peptide sequence and lacking an HA peptide sequence

(19) United States
(12) Patent Application Publication
(10) Pub. No.: US 2014/0303093 A1
(43) Pub. Date: Oct. 9, 2014

(54) MICRO-UTROPHIN POLYPEPTIDES AND METHODS

(71) Applicants: James M. Ervasti, Shoreview, MN (US); Hanke Heun-Johnson, Los Angeles, CA (US)

(72) Inventors: James M. Ervasti, Shoreview, MN (US); Hanke Heun-Johnson, Los Angeles, CA (US)

(73) Assignee: Regents of the University of Minnesota, Minneapolis, MN (US)

(21) Appl. No.: 14/131,882

(22) PCT Filed: Jul. 12, 2012
(86) PCT No.: PCT/US2012/046375

§ 371 Date: Mar. 27, 2014

Related U.S. Application Data
(60) Provisional application No. 61/506,706, filed on Jul. 12, 2011.

(51) Int. Cl. C07K 14/47

(52) U.S. Cl. CPC C07K 14/4708
USPC 514/21.2; 530/350; 536/23.4

(57) ABSTRACT

Described herein are polypeptides, polynucleotides and methods involving a μ-utrophin region or an anti-dystrophinopathic fragment thereof operationally linked to a second region effective to transduce the fusion protein into mammalian muscle cells.

### CROSS-REFERENCE TO RELATED APPLICATION

This application claims priority to U.S. Provisional Patent Application Ser. No. 61/506,706, filed Jul. 12, 2011, which is incorporated herein by reference in its entirety.

### BACKGROUND

Duchenne muscular dystrophy (DMD) is a lethal X-linked disorder affecting approximately one in every 3500 born males (Emery, 1991 Neuromuscul Disord 1:19-29). DMD results from the loss of dystrophin, a 427 kDa protein localized to the subsarcolemmal space of cardiac and skeletal muscle cells (Hoffman et al., 1987 Cell 51:919-28). Dystrophin deficiency is associated with increased sarcolemmal permeability, cycles of muscle cell death and regeneration, and muscle weakness that eventually causes death due to respiratory and/or cardiac failure (Emery, 2002 Lancet 359:687-95). Biochemical studies reveal dystrophin to be associated with a multi-subunit complex, the dystrophin-glycoprotein complex (DGC) (Ervasti, 2007 Biochim Biophys Acta 1772:108-171; Blake, 2002 Physiol Rev 82:291-329). Dystrophin forms a biochemically stable complex with the membrane-embedded dystroglycan and sarcoglycan/sarcospan subcomplexes, as well as cytoplasmic dystrobrevins and syntrophins. Dystrophin also interacts with actin filaments (Ervasti, 2007 Biochim Biophys Acta 1772:108-17), intermediate filaments (Bhosle et al., 2006 Biochem Biophys Res Commun 346:768-77), and microtubules of the cytoskeleton (Prins et al., 2009 J Cell Biol 186:363-9). Based on the structure of dystrophin, its interactions with other cellular constituents, and pathologies that manifest when it is absent, dystrophin is thought to primarily function as a structural protein that stabilizes the sarcolemma during mechanical activity (Petrof et al., 1993 Proc Natl Acad Sci USA 90:3710-4). Despite intensive effort by many laboratories investigating a variety of elegant therapeutic strategies, there is presently no cure or effective treatment that can alleviate the devastating progression of DMD.

### SUMMARY

In one aspect, this disclosure describes an isolated μ-utrophin (μUtr) polypeptide. Generally, the polypeptide includes a μ-utrophin region or an anti-dystrophinopathic fragment thereof operationally linked to a second region effective to transduce the fusion protein into mammalian muscle cells, with the proviso that the isolated polypeptide does not include SEQ ID NO:1, a known FLAG affinity tag.

In another aspect, this disclosure describes an isolated nucleic acid expression construct encoding a polypeptide. Generally, the nucleic acid expression construct includes a first nucleic acid region that encodes a μ-utrophin polypeptide or an anti-dystrophinopathic fragment thereof, and a second nucleic acid region that encodes an amino acid sequence effective to transduce the μ-utrophin polypeptide into mammalian muscle cells operationally linked to the first nucleic acid region, with the proviso that the polypeptide does not include SEQ ID NO:1.

In another aspect, this disclosure describes a composition that includes an isolated μUtr polypeptide or a pharmaceutically suitable salt thereof in combination with a pharmaceutically suitable carrier.

In another aspect, this disclosure describes a method of treating dystrophinopathies in a subject. Generally, the method includes administering to a subject in need such treatment an anti-dystrophinopathic amount of an isolated μUtr polypeptide.

In another aspect, this disclosure describes a method of isolating the μUtr polypeptide described above. Generally, the method includes receiving a sample comprising the μUtr polypeptide, performing cation exchange chromatography on at least a portion of the sample, and

recovering the polypeptide at a purity of at least 86% and a yield of at least 90%.

In yet another aspect, this disclosure describes a method of isolating a polypeptide that possesses a net negative charge. Generally, the method includes obtaining a sample that includes a fusion polypeptide, in which the fusion polypeptide includes the polypeptide possessing a net negative charge and a positively charged tag comprising at least 12 amino acids, wherein at least one of the following is true: the positively charged tag comprises at least one non-arginine amino acid residue and/or the positively charged tag is located at the N-terminal of the fusion polypeptide; and performing cation exchange chromatography on at least a portion of the sample.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Purification of TAT-μUtr by cation-exchange chromatography. Coomassie blue-stained gels loaded with lysates (T) from Sf9 cells expressing TAT-μUtr (a), FLAG-TAT-μUtr (b), or FLAG-μUtr (c), the voids (V) after passage over SP Sepharose, column wash (W), and fractions eluted with the indicated [NaCl] gradient. (d) A Coomassie blue-stained gel or corresponding western blot loaded with equal amounts of purified FLAG-TAT-μUtr and TAT-μUtr. The western blot was blotted with a rabbit polyclonal antibody to the FLAG epitope and utrophin-specific monoclonal antibody 8A4. The molecular weight standards in kDa are indicated on the left of panels (a) and (d).

FIG. 2. TAT-μUtr stability in vivo. (a) A quantitative comparison of the decay in fluorescently-labeled FLAG-TAT-μUtr and TAT-μUtr in whole-body (WB), quadriceps (Q) and liver (L) as a function of time after a single IP injection. Whole body fluorescence was normalized to auto-fluorescence. Tissue lysates were resolved on SDS-gels and transferred to nitrocellulose. Tissue fluorescence was normalized to protein load by densitometry of the Coomassie blue-stained gels after transfer (n=2 per time point). (b) Western blots of the μUGC constituents syntrophin (Syn), α-sarcoglycan (α-SG), and β-dystroglycan (β-DG) in the quadriceps muscle enriched from detergent-solubilized skeletal muscle by WGA Sepharose chromatography. The dihydropyridine receptor (DHPR) was used as a loading control. The molecular weight standards are in kDa.

FIG. 3. Restoration of dystrophin-associated proteins to the sarcolemma following long-term administration of TAT-μUtr in mdx mice. Mice were administered PBS or TAT-μUtr twice weekly for 13.5 weeks. Shown are cryosections of quadriceps muscle stained with antibodies to laminin (Lam), utrophin (Utr), α-dystroglycan (α-DG), α-sarcoglycan (α-SG), dystrobrevin (DB), or neuronal nitric oxide synthase (nNOS). Scale bar=100 μm.

FIG. 4. Physiological improvements with short- and long-term TAT-μUTR treatment. mdx mice were treated short-term (2.5 weeks) or long-term (13.5) weeks with TAT-μUTR or PBS. Serum was analyzed for (a) creatine kinase activity (U/L) in mice treated with PBS (18055±4339; n=8) or TAT-μUTR (6177±1005; n=11; P=0.007). Muscle strength was analyzed by (b) grip strength normalized to body mass (g/g) in PBS−(2.5±0.2; n=13) and TAT-μUTR-treated mice (3.1±0.1; n=14; P=0.038). Isolated EDL muscles were tested for (c) specific force (P=0.662) and (d) the percentage of force loss during eccentric contractions in PBS (64±4; n=14) and TAT-μUTR (34±6; n=14; P<0.001) groups. Data are presented as means±SEM. All data were analyzed with a 2-way ANOVA. Significant interactions were not detected with any variable, and P-values represent the main effects of treatment. Circles=PBS; squares=TAT-μUTR. *Signifies main effect of treatment. **Signifies main effect of time.

FIG. 5. Skeletal muscle histology is not improved with TAT-μUTR. mdx mice were treated short-term (2.5 weeks) or long-term (13.5) weeks with TAT-μUTR or PBS. Cryosections were prepared from quadriceps, EDL, and diaphragm muscles and were stained with hematoxylin and eosin-phloxine to quantify centrally nucleated fibers (CNFs). Presented in (a) are representative images (200×) from mdx mice that were treated long-term with PBS- and TAT-μUTR. Scale bar=100 μm. The percentage of CNFs were not different with treatment in (b) the quadriceps muscle (P=0.148) or (c) the EDL muscle (P=0.627). Data are presented as means±SEM. All data were analyzed with a 2-way ANOVA. Significant interactions were not detected with any variable, and P-values represent the main effects of treatment. Circles=PBS; squares=TAT-μUTR. **Signifies main effect of time.

### DETAILED DESCRIPTION OF ILLUSTRATIVE EMBODIMENTS

The loss of dystrophin causes Duchenne muscular dystrophy and some forms of dilated cardiomyopathy. Recently, we demonstrated in short-term proof-of-concept trials that a FLAG-tagged TAT-μ-utrophin fusion protein provides an effective direct protein-replacement therapy for striated muscle diseases caused by dystrophin deficiency. Although the protein construct tested contained a FLAG tag to facilitate purification, the tag poses problems for advancing protein replacement toward clinical trials. Here, we report the generation of a FLAG-less TAT-μ-utrophin construct. We have developed a rapid one-step purification using cation-exchange chromatography that provides the basis for a broadly applicable and scalable method to purify TAT-conjugated therapeutics. We also demonstrate that FLAG-less TAT-μ-utrophin is effective in reducing the dystrophic phenotypes of dystrophin-deficient mdx mice, even when administered twice weekly over a period of three weeks or three months. Our new results establish the long-term efficacy of a TAT-μ-utrophin construct that can be expressed and purified in a scalable manner using inexpensive chromatography supports.

In one aspect, this disclosure describes a FLAG-less TAT-μ-utrophin construct. In another aspect, this disclosure describes a rapid one-step purification using cation-exchange chromatography that may provide a basis for a broadly applicable and scalable method to purify fusion polypeptides that include a positively-charged tag. In one specific example, such a method may provide a basis for purifying TAT-fusion therapeutics such as, for example, TAT-μ-utrophin.

As used herein, the following terms shall have the indicated meanings:

“TAT” refers to the highly basic protein transduction domain (PTD) of the Human Immunodeficiency Virus (HIV)-1 Trans-Activator of Transcription protein, or a fragment thereof that induces transduction of a polypeptide containing the TAT amino acid into a mammalian cell.

“μ-utrophin” refers to a truncated form of utrophin in which designated spectrin-like repeats are deleted. An exemplary μ-utrophin in which spectrin-like repeats 4-21 are deleted is depicted in SEQ ID NO:9, beginning at amino acid 14 of SEQ ID NO:9.

“FLAG-tag” refers to a polypeptide protein tag (DYKDDDDK, SEQ ID NO:1) that can be added to a protein using, for example, routine recombinant DNA technology. It can be used for affinity chromatography separation of recombinantly-produced polypeptides that possess the tag.

“Ameliorate” refers to any reduction in the extent, severity, frequency, and/or likelihood of a symptom or clinical sign characteristic of a particular condition.

“Prophylactic” and variations thereof refer to a treatment that limits, to any extent, the development and/or appearance of a symptom or clinical sign characteristic of a condition. Prophylactic treatments are often initiated before the manifestation of a symptom or clinical sign characteristic of the condition.

“Sign” or “clinical sign” refers to an objective physical finding relating to a particular condition capable of being found by one other than the patient.

“Symptom” refers to any subjective evidence of disease or of a patient's condition.

“Therapeutic” and variations thereof refer to a treatment that ameliorates one or more existing symptoms or clinical signs associated with a condition.

“Treat” or “treatment” or any variation thereof refers to reducing, ameliorating, or resolving, to any extent, the symptoms or signs related to a condition.

The terms “comprises” and variations thereof do not have a limiting meaning where these terms appear in the description and claims; unless otherwise specified, “a,” “an,” “the,” and “at least one” are used interchangeably and mean one or more than one; the term “and/or” means one or all of the listed elements or a combination of any two or more of the listed elements; and the recitations of numerical ranges by endpoints include all numbers subsumed within that range (e.g., 1 to 5 includes 1, 1.5, 2, 2.75, 3, 3.80, 4, 5, etc.).

Utrophin is a widely expressed autosomal gene product with high sequence similarity to dystrophin (Tinsley et al., 1992 Nature 360:591-3). Utrophin is distributed throughout the sarcolemma in fetal and regenerating muscle, but is down-regulated in normal adult muscle and restricted to the myotendinous and neuromuscular junctions (Blake et al., 1996 Brain Pathol 6:37-47). Utrophin is thought to compensate for dystrophin-deficiency because mice lacking both dystrophin and utrophin exhibit a more severe, DMD-like phenotype (Deconinck et al., 1997 Cell 90:717-27; Grady et al., 1997 Cell 90:729-38), while overexpression of utrophin rescues the dystrophic phenotype in mdx mice (Tinsley et al., 1998 Nat Med 4:1441-4). Thus, various approaches that involve overexpression have been investigated as potential therapies for DMD (Khurana and Davies, 2003 Nat Rev Drug Discov 2:379-90; Krag et al., 2004 Proc Natl Acad Sci USA 101:13856-60; Odom et al., 2008 Mol Ther 16:1539-45; Squire et al., 2002 Hum Mol Genet 11:3333-44).

The highly basic protein transduction domain (PTD) of the HIV-1 TAT protein has been used to effectively mediate delivery of various cargoes into cells of many tissues (Schwarze et al., 1999 Science 285:1569-72; Haase et al., 2006 Faseb J 20:865-73). Systemic delivery of a recombinant fusion protein encoding a FLAG-tagged TAT-μ-utrophin (FLAG-TAT-μUtr) can transduce skeletal muscle in vivo, assemble into a μ-utrophin-glycoprotein complex (μUGC), and improve several parameters of the dystrophic phenotype in mdx mice (Sonnemann et al., 2009 PLoS Med 6:e1000083). While direct protein replacement may be feasible in human patients with DMD, the acidic, amino-terminal FLAG tag (DYKDDDDK, SEQ ID NO:1) that facilitates purification of the fusion protein may reduce the suitability of the FLAG-TAT-μUtr construct for protein replacement therapy. The FLAG epitope was originally designed to be highly immunogenic (Hopp et al., 1988 Bio/Technology 6:1204-1210). Moreover more recent data suggest that a proximally-tethered acidic polypeptide, such as the FLAG tag, can inhibit TAT-mediated transduction (Aguilera et al., 2009 Integr Biol (Camb) 1:371-81; Jiang et al., 2004 Proc Natl Acad Sci USA 101:17867-72; Olson et al., 2009 Integr Biol (Camb) 1:382-93). To address these potential problems, we have generated a FLAG-less TAT-μUtr (TAT-μUtr) construct and show that it can be isolated in one step using cation-exchange chromatography to the same degree of purity as FLAG-TAT-μUtr. We also report studies demonstrating efficacy for TAT-μUtr in both short and long-term trials of mdx mice.

Generating a FLAG-less TAT-μUtr required constructing a baculovirus construct encoding FLAG-TAT-μUtr and, due to elimination of the FLAG epitope, which was used for affinity purification of FLAG-tagged μUtr, necessitated development of an alternative purification strategy.

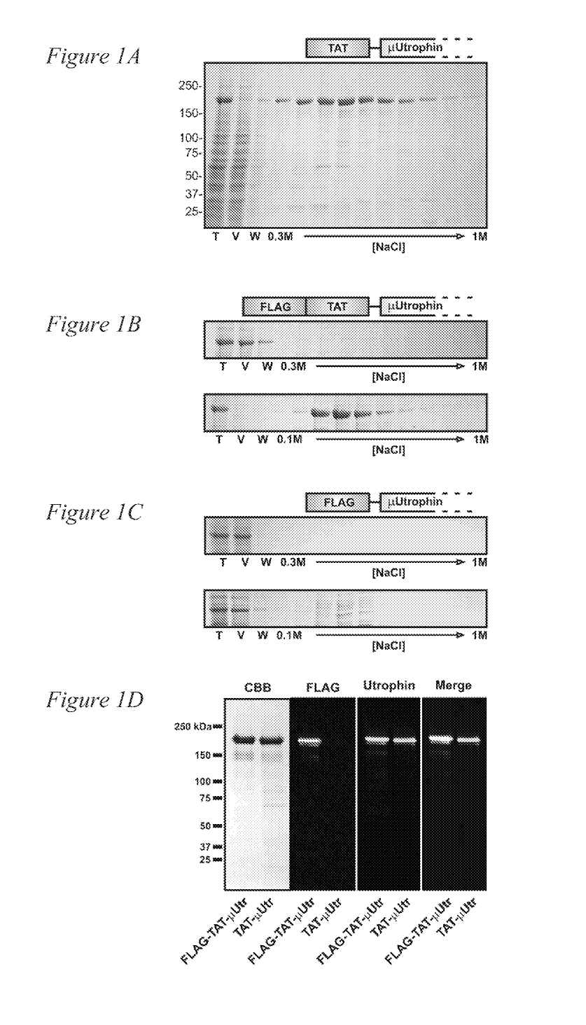

Baculovirus-infected Sf9 cell lysates containing TAT-μUtr, FLAG-TAT-μUtr, or FLAG-μUtr were loaded onto SP-Sepharose columns, and the columns eluted with linear NaCl gradients (FIG. 1a-1c). TAT-μUtr was quantitatively removed from the SP-Sepharose void, even when the lysate contained 0.3 M NaCl, and eluted in a highly purified state at a NaCl concentration of 0.7 M (FIG. 1a). Under the same conditions, FLAG-TAT-μUtr failed to bind to the SP-Sepharose, but could be purified if the NaCl concentration in the insect cell lysates and initial column wash was lowered to 0.1 M (FIG. 1b). We also determined that the TAT sequence was required for high salt purification of TAT-μUtr on SP-Sepharose, because FLAG-μUtr failed to bind SP-Sepharose in the presence of either 0.1 M NaCl or 0.3 M NaCl (FIG. 1c). The relative purities of SP-Sepharose-purified TAT-μUtr and anti-FLAG M2-agarose-purified FLAG-TAT-μUtr were similar (93%), as assessed by densitometric analysis of Coomassie blue-stained gels and western blot analysis (FIG. 1d). These data demonstrate that a FLAG-less TAT-μUtr can be efficiently and economically purified using a conventional chromatography support.

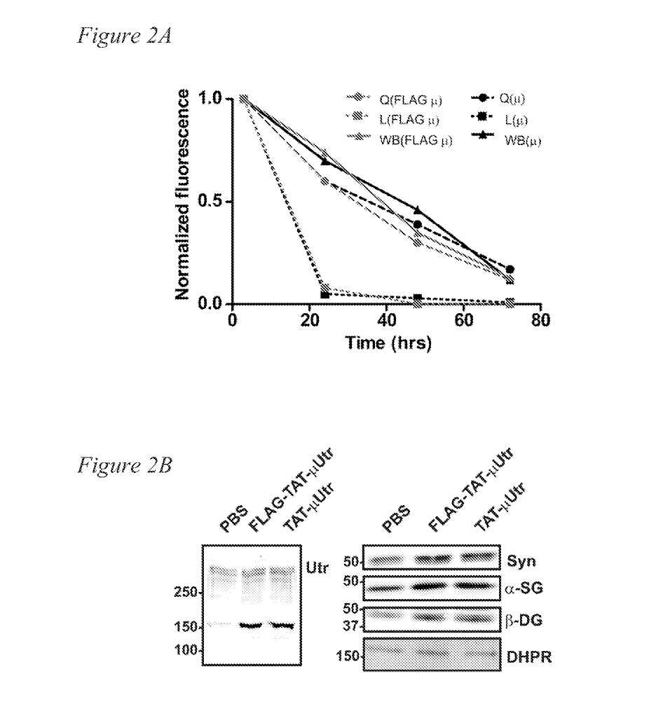

To compare the relative stabilities of TAT-μUtr with FLAG-TAT-μUtr in vivo, we administered one injection (8.5 μg/g body mass) of each protein labeled with an infrared-excitable fluorescent dye into mdx mice. Mice were sacrificed and imaged for whole body fluorescence at 3 hours, 24 hours, 48 hours, or 72 hours after the injection. Tissues that were collected from the mice at each time point were imaged at the protein level after SDS-PAGE (FIG. 2a). TAT-μUtr and FLAG-TAT-μUtr yielded similar whole-body fluorescence intensities and rates of decay. This mirrored the decay rate in quadriceps muscle, while decay of both proteins in the liver was more rapid (FIG. 2a).

Because FLAG-TAT-μUtr was previously shown to form a biochemically-stable complex with other members of the utrophin glycoprotein complex (UGC) (Sonnemann et al., 2009 PLoS Med 6:e1000083), we performed wheat germ agglutinin (WGA) chromatography on detergent-solubilized muscle extracts from mdx mice treated with TAT-μUtr or FLAG-TAT-μUtr. Western blot analysis demonstrated that both TAT-μUtr and FLAG-TAT-μUtr co-purified with increased amounts of dystrophin-associated proteins, compared to PBS controls, while the amount of co-purifying endogenous utrophin was unchanged (FIG. 2b). These data suggest that TAT-μUtr is as effective as FLAG-TAT-μUtr in transducing mdx muscle and integrating into a μUGC.

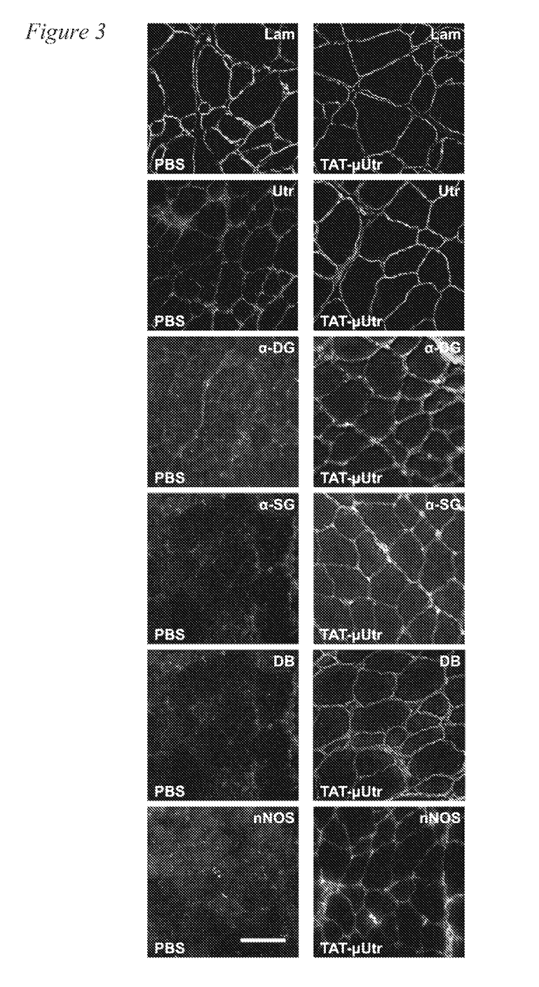

For direct protein replacement to become clinically useful, it must demonstrate both short-term and long-term efficacy. Therefore, we tested TAT-μUtr (8.5 mg/kg body mass) by injecting the construct twice weekly for either 2.5 weeks or 13.5 weeks in mdx mice, and compared them to PBS-treated mdx mice. Injections started at P18 and continued until the mice were either 5 weeks old or 16 weeks old. Because the loss of dystrophin expression leads to a destabilization and concomitant loss of other dystrophin-glycoprotein complex members from the muscle cell membrane (Blake, 2002 Physiol Rev 82:291-329), we first examined whether long-term administration of TAT-μUtr restored the μUGC at the sarcolemma. We examined quadriceps muscle cryosections with primary antibodies to utrophin and observed intense staining along the periphery of muscle cells in TAT-μUtr-treated mice, with light staining in PBS-injected samples (FIG. 3). The staining pattern for TAT-μUtr was appropriately targeted to the subsarcolemmal space, consistent with our previous short-term experiments (Sonnemann et al., 2009 PLoS Med 6:e1000083). Immunofluorescence analyses using antibodies against α-dystroglycan and α-sarcoglycan demonstrated intense staining along the periphery of muscle fibers from TAT-μUtr-treated mice, while these proteins were barely detectable on cryosections from PBS-injected mice. In addition, the intracellular proteins dystrobrevin and nNOS were also localized to the cell periphery of treated muscle, suggesting that long-term administration of TAT-μUtr maintained a stable μUGC at the sarcolemma.

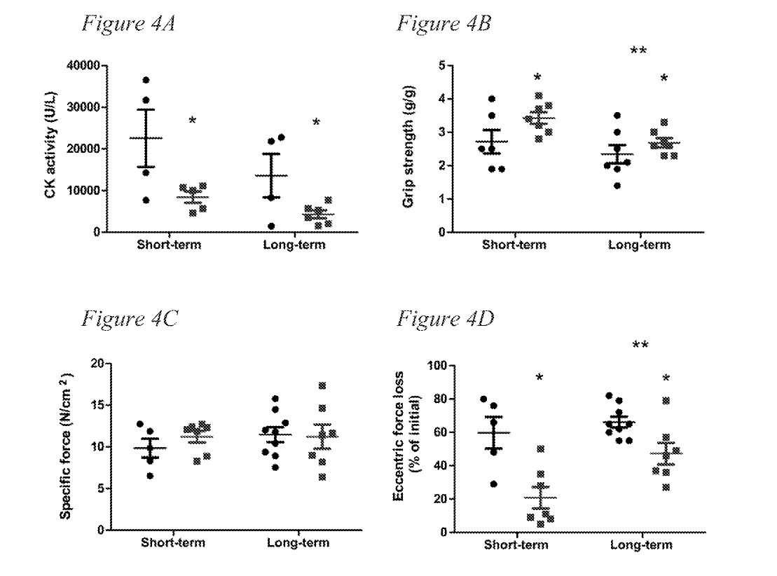

Twice-weekly intraperitoneal administration of TAT-μUtr into mdx mice conferred significant improvement in several parameters of the dystrophic phenotype compared to PBS-injected littermates (FIG. 4). Muscle membrane stability was also significantly improved, with TAT-μUtr-treated mice having serum creatine kinase levels that were 66% lower as compared to PBS-injected mice (FIG. 4a), independent of length of treatment. We also employed an in vivo measure of muscle strength (grip strength), and found that TAT-μUtr-treated mice exhibited 21% greater voluntary forelimb muscle strength (FIG. 4b). Extensor digitorum longus (EDL) muscles were also tested to determine electrically-evoked maximal isometric force and susceptibility to injury from eccentric contractions. Specific force (FIG. 4c) was not significantly improved with treatment. However, TAT-μUtr-treated mice were significantly protected from eccentric contraction-induced injury, since they generated 47% more force than PBS-injected mice after five eccentric contractions (FIG. 4d). Thus, short-term and long-term studies demonstrate that TAT-μUtr improves membrane integrity and muscle function following high-force contractions that usually induce injury.

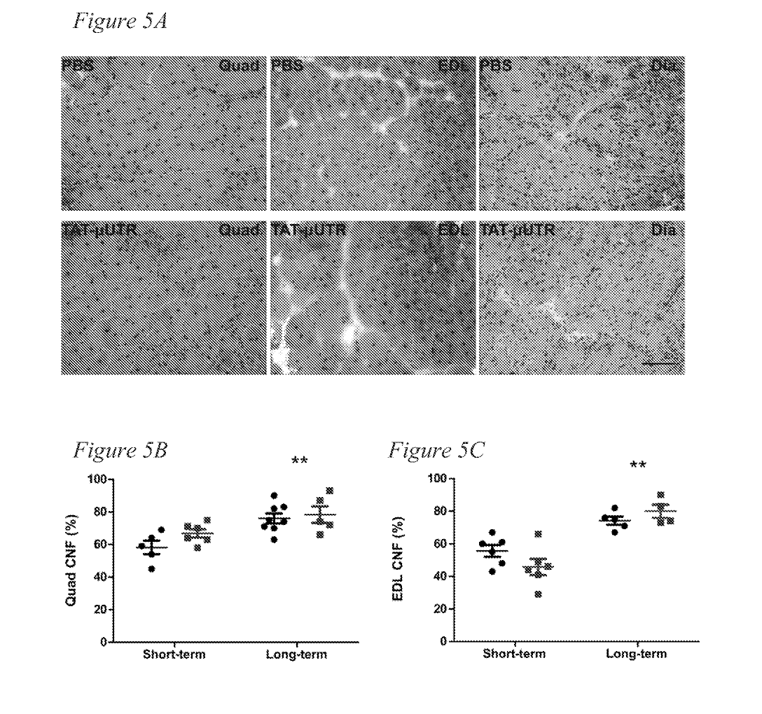

Cyrosections from quadriceps, EDL, and diaphragm muscles were prepared and stained with hematoxylin and eosin-phloxine to quantify the number of centrally-nucleated fibers, an indicator of muscle fiber degeneration/regeneration (FIG. 5a). We did not observe an improvement in the percentage of centrally-nucleated fibers in TAT-μUtr-treated quadriceps or EDL muscles as compared to PBS-injected mdx controls (FIG. 5b and FIG. 5c). Qualitative examination of diaphragm muscle strips showed centrally-nucleated fibers, fibrosis, muscle fiber size variability, and the presence of inflammatory cells in both PBS-treated and TAT-μUTR-treated samples. Unlike FLAG-TAT-μUTR (Sonnemann et al., 2009 PLoS Med 6:e1000083), histological improvement of skeletal muscles did not occur with short-term or long-term treatments of TAT-μUTR.

We recently demonstrated that systemic delivery of a recombinant fusion protein encoding FLAG-tagged TAT-μ-utrophin could significantly improve several phenotypic parameters of dystrophy in mdx mice (Sonnemann et al., 2009 PLoS Med 6:e1000083). Our current study builds on our previous findings and addresses three important issues.

First, while the FLAG-tag can often facilitate purification of recombinant proteins via immuno-affinity chromatography, its inclusion in biotherapeutics is problematic due to its inherent immunogenicity (Hopp et al., 1988 Bio/Technology 6:1204-1210). Furthermore, recent studies (Aguilera et al., 2009 Integr Biol (Camb) 1:371-81; Jiang et al., 2004 Proc Natl Acad Sci USA 101:17867-72; Olson et al., 2009 Integr Biol (Comb) 1:382-93) suggest that a covalently linked acidic polypeptide—e.g., a FLAG-tag—can inhibit TAT-mediated transduction into cells. Therefore, we generated a construct lacking the FLAG tag and successfully purified TAT-μUtr protein using an inexpensive cation-exchange chromatography protocol that should be applicable to many TAT-fusion proteins.

Second, we show that both short-term and long-term treatment of mdx mice with TAT-μUtr significantly improved several phenotypic parameters of muscular dystrophy. After twice-weekly administration of TAT-μUtr to mdx mice for 3-14 weeks, several proteins of the DGC/UGC were upregulated in TAT-μUtr-treated skeletal muscles, including α-sarcoglycan, syntrophin, β-sarcoglycan, α-dystroglycan, dystrobrevin, and nNOS. We measured significant reductions in serum creatine kinase, improved grip strength, and less susceptibility to eccentric contraction-induced injury, as compared to PBS-injected littermates. In the current study, serum creatine kinase and eccentric force loss were improved 47%-66% in mdx mice treated with TAT-μUtr. These results extend previous results using the short-term treatment paradigm in mdx mice showing ˜50% improvements in these same parameters with FLAG-TAT-μUtr (Sonnemann et al., 2009 PLoS Med 6:e1000083). Based on the sum of our results, we conclude that the presence of the FLAG-epitope has no impact on the stability, transduction, or therapeutic efficacy of TAT-μUtr in ameliorating strength deficits and membrane permeability associated with dystrophinopathy in mice.

Thus, in one aspect, this disclosure describes polypeptide comprising a μUtr polypeptide. As used herein, “polypeptide” refers to a polymer of amino acids linked by peptide bonds. Thus, for example, the terms peptide, oligopeptide, protein, and enzyme are included within the definition of polypeptide. This teem also includes post-expression modifications of the polypeptide, such as glycosylations, acetylations, phosphorylations, and the like. The term polypeptide does not connote a specific length of a polymer of amino acids. A polypeptide may be isolatable directly from a natural source, or can be prepared with the aid of recombinant, enzymatic, or chemical techniques. In the case of a polypeptide that is naturally occurring, such a polypeptide is typically isolated. An “isolated” polypeptide is one that has been removed from its natural environment. For instance, an isolated polypeptide is a polypeptide that has been removed from the cytoplasm or from the membrane of a cell, and many of the polypeptides, nucleic acids, and other cellular material of its natural environment are no longer present. An “isolatable” polypeptide is a polypeptide that could be isolated from a particular source. A “purified” polypeptide is one that is free, to any specified degree, from other components with which they are naturally associated. Polypeptides that are produced outside the organism in which they naturally occur—e.g., through chemical or recombinant means—are considered to be isolated and purified by definition, since they were never present in a natural environment. As used herein, a “polypeptide fragment” refers to a portion of a polypeptide. A polypeptide fragment may result from digestion of a polypeptide with a protease or may be produced using recombinant, enzymatic, or chemical techniques.

As used herein, a “μUtr polypeptide” is a utrophin polypeptide in which at least one spectrin-like repeat of the native form of the utrophin polypeptide is deleted. Exemplary μUtr polypeptides are depicted in SEQ ID NO:3 (beginning at amino acid 41 of SEQ ID NO:3), SEQ ID NO:5 (beginning at amino acid 41 of SEQ ID NO:5), SEQ ID NO:7 (beginning at amino acid 33 of SEQ ID NO:7), SEQ ID NO:9 (beginning at amino acid 14 of SEQ ID NO:), SEQ ID NO:10 (beginning at amino acid 31 of SEQ ID NO:10) and SEQ ID NO:11 (beginning at amino acid 13 of SEQ ID NO:11).

A μUtr polypeptide may be derived from a variety of species of mammals including, but not limited to, humans, non-human primates, rats, mice, cows, pigs, dogs, etc.

A μUtr polypeptide also may include a “biologically active analog” of a reference μUtr polypeptide. Functional activity of a μUtr polypeptide can be assessed using the various assays described herein as well as other assays well known to one with ordinary skill in the art. A modulation in functional activity, including the stimulation or the inhibition of functional activity, can be readily ascertained by the various assays described herein, and by assays known to one of skill in the art.

A modulation in a functional activity can be quantitatively measured and described as a percentage of the functional activity of a comparable control. The functional activity of a μUtr polypeptide may exhibit modulation of a μUtr activity that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% of the activity of a suitable control.

For example, the stimulation of a functional activity of a μUtr polypeptide can be quantitatively measured and described as a percentage increase of the functional activity of a comparable control. Stimulation of a functional activity of a μUtr polypeptide includes a stimulation that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% greater than the activity of a suitable control.

As another example, inhibition of a functional activity of a μUtr polypeptide can be quantitatively measured and described as a percentage of the functional activity of a comparable control. Inhibition of a functional activity of a μUtr polypeptide includes an inhibition that is no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 99%, or less than 100% of the activity of a suitable control.

A “biologically active analog” of a μUtr polypeptide includes polypeptides having one or more amino acid substitutions that do not eliminate a functional activity. Substitutes for an amino acid in a biologically active analog of a μUtr polypeptide may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Examples of such preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH₂. Likewise, a biologically active analog of a μUTR polypeptide can include an addition and/or a deletion of one or more contiguous or noncontiguous amino acids that do not eliminate a functional activity of a μUTR polypeptide.

An addition of one or more contiguous amino acids may provide a desired functionality to the μUtr polypeptide. For example, a μUtr polypeptide can include a TAT sequence (e.g., YGRKKRRQRRR, shown in amino acids 11-21 of SEQ ID NO:3, amino acids 11-21 of SEQ ID NO:5, amino acids 3-13 of SEQ ID NO:7, amino acids 3-16 of SEQ ID NO:9, amino acids 2-12 of SEQ ID NO:10, and SEQ ID NO:11) or a HA sequence (e.g., YPYDVPDYA, shown in amino acids 29-37 of SEQ ID NO:3, amino acids 29-37 of SEQ ID NO:5, amino acids 21-29 of SEQ ID NO:7, and amino acids 20-28 of SEQ ID NO:10). A TAT and/or HA addition can provide function such as, for example, transducing the μUtr polypeptide into mammalian muscle cells and/or facilitate purification of the μUtr polypeptide. Exemplary embodiments of μUtr polypeptides include polypeptides that include a TAT and a HA addition (e.g., SEQ ID NO:10) as well as polypeptides that include, for example, a TAT addition but no HA addition (e.g., SEQ ID NO:11).

Despite the variety of possible amino acid additions, a μUTR polypeptide as described herein expressly lacks a FLAG-tag (SEQ ID NO:1).

A “biologically active analog” of a μUtr polypeptide includes “fragments” and “modifications” of a μUtr polypeptide. As used herein, a “fragment” of a μUtr polypeptide means a μUtr polypeptide that has been truncated at the N-terminus, truncated at the C-terminus, possesses one or more deletions of contiguous amino acids (e.g., all or a portion of a spectrin-like repeat), or any combination thereof, that possesses anti-dystrophinopatic activity.

A “modification” of a μUtr polypeptide includes μUtr polypeptides or fragments thereof chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like. A modified μUtr polypeptide may retain the biological activity of the unmodified polypeptide or may exhibit a reduced or increased biological activity.

A μUtr polypeptide or a biologically active analog thereof may be recombinantly produced, chemically synthesized, or enzymatically synthesized.

A μUtr polypeptide can include a polypeptide with “structural similarity” to the μUtr portions of the polypeptides depicted in SEQ ID NO:3, SEQ ID NO:5, SEQ ID NO:7, SEQ ID NO:9, SEQ ID NO:10, SEQ ID NO:11, and/or fragments thereof. As used herein, “structural similarity” refers to the identity between two polypeptides. For polypeptides, structural similarity is generally determined by aligning the residues of the two polypeptides (for example, a candidate polypeptide and the μUtr portion of the polypeptide of SEQ ID NO:3, SEQ ID NO:5, SEQ ID NO:7, SEQ ID NO:9, SEQ ID NO:10, SEQ ID NO:11, or a relevant functional fragment thereof) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the μUtr polypeptide. A candidate polypeptide can be produced using recombinant techniques, or chemically or enzymatically synthesized.

A pair-wise comparison analysis of μUtr polypeptide sequences can carried out using the BESTFIT algorithm in the GCG package (version 10.2, Madison Wis.). Alternatively, polypeptides may be compared using the Blastp program of the BLAST 2 search algorithm, as described by Tatiana et al., (1999 FEMS Microbiol Lett, 174, 247-250), and available on the world wide web at ncbi.nlm.nih.gov/BLAST/. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids and “similarity” refers to the presence of not only identical amino acids but also the presence of conservative substitutions.

A μUtr polypeptide can include a polypeptide exhibiting at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference μUtr amino acid sequence.

Alternatively, as used herein, reference to a μUtr polypeptide and/or reference to the amino acid sequence of one or more SEQ ID NOs can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Amino acids essential for the function of μUTR polypeptides can be identified according to procedures known in the art, such as site-directed mutagenesis or alanine-scanning mutagenesis (Cunningham and Wells, 1989 Science 244: 1081-1085; Bass et al., 1991 Proc. Natl. Acad. Sci. USA 88: 4498-4502).

As noted above, a μUtr polypeptide can include an addition such as, for example, a tag that can provide one or more additional functions to the μUtr polypeptide. Examples of such tags include, for example, the TAT protein transduction domain and/or a HA tag. The highly basic nature of the TAT protein transduction domain also can function as a tag that facilitates binding of a polypeptide that contains the TAT protein transduction domain to, for example, a cation exchange column. Thus, a fusion polypeptide that includes a TAT tag may be isolatable in a scalable, single-step process using cation exchange chromatography.

Alternatively, additions other than a TAT tag can provide the basis for purification using cation exchange chromatography. In some embodiments, an amino acid addition having at least 12 amino acids and a net positive charge also can provide the basis for purification using cation exchange chromatography. In certain embodiments, such a positively-charged tag can have at least one non-arginine amino acid residue. In other embodiments, the positively-charged tag can be positioned at the N-terminal end of the μUtr polypeptide.

A μUtr polypeptide lacking a positively-charged tag such as, for example, a TAT tag, does not significantly bind to cation exchange column. In contrast, a μUtr polypeptide that includes a positively-charged tag—e.g., a tagged μUtr such as, for example, a TAT-μUtr polypeptide—not only binds to a cation exchange column, but permits recovery of significantly purified μUtr. For example, a TAT tag—and, therefore, a TAT-μUtr polypeptide—may permit a recovery of the μUtr polypeptide with a yield of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. As used herein, yield relates to the mass of a compound—e.g., a tagged μUtr such as, for example, TAT-μUtr—that is retained after a sample containing the compound is subjected to one or more preparatory and/or analytical procedures. Yield is expressed as the percentage of the mass of the compound in a sample that is successfully retained following the preparatory and/or analytical procedure or procedures. In certain embodiments, a μUtr polypeptide may be recovered with a yield of at least 90%.

Moreover, a positively-charged tag such as, for example, a TAT tag can provide recovery of μUtr polypeptide to a purity if at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. As used herein, “purity” is a quantifiable characteristic that refers to the relative amount of a compound of interest in a composition—e.g., a tagged μUtr such as, for example, TAT-μUtr—compared to other components of the composition. The relative amounts may be expressed in terms of the relative number of molecules (e.g., parts per hundred), in terms of molar equivalents, or in terms of relative mass. In certain embodiments, the μUtr polypeptide may be recovered to a purity of at least 86%. In other embodiments, the μUtr polypeptide may be recovered to a purity of at least 93%.

In some embodiments, the use of a positively-charged tag can permit that recovery of a compound of interest—e.g., tagged μUtr such as, for example, TAT-μUtr—that combines a specified yield and a specified purity. The specified yield may be any of the yield values set forth two paragraphs above; the specified purity may be any degree of purity set forth in the preceding paragraph. Thus, in one particular embodiment, recovery of a TAT-μUtr polypeptide using cation exchange chromatography can recover at least 90% of the TAT-μUtr polypeptide in the sample subjected to cation exchange chromatography to a purity of at least 86%.

Accordingly, a TAT-μ-utrophin polypeptide as described herein can possess multiple complementary functions. For example, the μ-utrophin can provide a therapeutic benefit of full-length utrophin in connection with conditions involving dystrophin deficiency. Moreover, because a μ-utrophin polypeptide is smaller than the full-length utrophin protein, μ-utrophin polypeptides may be more easily delivered to target cells. In addition, a positively-charged tag such as, for example, a TAT region can provide dual functionality. First, the positively-charged tag can induce transduction of a polypeptide containing the tag into mammalian cells. Second, the positively-charged tag can provide a means by which a polypeptide that includes the tag can be economically isolated using cation exchange chromatography.

A μUtr polypeptide may be formulated in a composition along with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with a μUtr polypeptide without causing any undesirable biological effects or interacting in a deleterious manner with any of the other components of the pharmaceutical composition in which it is contained.

A μUtr polypeptide may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). It is foreseen that a composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the μUtr polypeptide into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A μUtr polypeptide may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

In another aspect, this disclosure describes method that, in general, involves administering an effective amount of a μUtr polypeptide to a subject in need of treatment involving administering a μUtr polypeptide.

The amount of μUtr polypeptide administered can vary depending on various factors including, but not limited to, the specific μUtr polypeptide being administered, the weight, physical condition, and/or age of the subject, and/or the route of administration. Thus, the absolute weight of the μUtr polypeptide included in a given unit dosage form can vary widely, and depends upon factors such as the species, age, weight and physical condition of the subject, as well as the method of administration. Accordingly, it is not practical to set forth generally the amount that constitutes an amount of the μUtr polypeptide effective for all possible applications. Those of ordinary skill in the art, however, can readily determine the appropriate amount with due consideration of such factors.

In some embodiments, the method can include administering sufficient μUtr polypeptide to provide a dose of, for example, from about 100 ng/kg to about 50 mg/kg to the subject, although in some embodiments the methods may be performed by administering μUtr polypeptide in a dose outside this range. In some of these embodiments, the method includes administering sufficient μUtr polypeptide to provide a dose of from about 10 μg/kg to about 10 mg/kg to the subject, for example, a dose of from about 100 μg/kg to about 1 mg/kg. In one particular embodiment, the method includes administering about 8.5 mg/kg to the subject.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the methods can include administering sufficient μUtr polypeptide to provide a dose of, for example, from about 0.01 mg/m² to about 10 mg/m².

In some embodiments, the μUtr polypeptide may be administered, for example, from a single dose to multiple doses per week, although in some embodiments the methods disclosed herein may be performed by administering the μUtr polypeptide at a frequency outside this range. In certain embodiments, the μUtr polypeptide may be administered from about once per month to about five times per week.

Thus, in yet another aspect, this disclosure describes a method that includes providing a composition comprising a μUtr polypeptide, wherein the composition is effective to ameliorate at least one symptom or clinical sign of a condition characterized, at least in part, by a dystrophin deficiency. Such conditions can include, for example, Duchenne muscular dystrophy and/or dilated cardiomyopathy. Typical symptoms and/or clinical signs that may be ameliorated by administering a μUtr polypeptide as described herein include, for example, sarcolemmal damage (assessed by, e.g., measuring serum creatine kinase activity), skeletal muscle weakness and fatigue (measured by, e.g., the six-minute walk test, grip test, or manual muscle testing), pulmonary insufficiency (e.g., maximal inspiratory and/or expiratory pressures, peak cough flow), and/or cardiac monitoring for a delay in progression to heart failure symptoms

In another aspect, this disclosure describes methods for making antibodies, for example, by either inducing the production of antibody in an animal or by recombinant techniques. The antibody produced includes antibody that specifically binds at least one μUtr polypeptide or fragment thereof. Thus, in a related aspect, this disclosure describes antibody that specifically binds to a μUtr polypeptide or fragment thereof, and compositions including such antibodies.

The method may be used to produce an antibody composition that specifically binds a μUtr polypeptide. As used herein, an antibody that can “specifically bind” a μUtr polypeptide is an antibody that interacts with the epitope of the μUtr polypeptide or interacts with a structurally related epitope and/or having a differential or a non-general (i.e., non-specific) affinity, to any degree, for a μUtr polypeptide. In some embodiments, an antibody composition can include polyclonal antibody raised against a μUtr polypeptide. In other embodiments, an antibody composition can include one or more monoclonal antibodies raised against a μUtr polypeptide. In still other embodiments, an antibody of the antibody composition may be synthesized through recombinant or synthetic methods. In some embodiments, the method may result in the production of antibody that specifically binds to a μUtr polypeptide but does not specifically bind to full-length utrophin.

In another aspect, this disclosure describes a μUtr polynucleotide—i.e., an isolated polynucleotide that encodes at least a portion of a μUtr polypeptide. Examples of a μUtr polynucleotide include an isolated polynucleotide that encodes an amino acid that includes the μUtr amino acid sequence of, for example, SEQ ID NO:3, SEQ ID NO:5, SEQ ID NO:7, SEQ ID NO:9, SEQ ID NO:10, or SEQ ID NO:11, or the complements of such polynucleotide sequences. Other examples of a μUtr polynucleotide include an isolated polynucleotide that hybridizes, under standard hybridization conditions, to a polynucleotide that encodes an amino acid sequence that includes the μUtr amino acid sequence of, for example, SEQ ID NO:3, SEQ ID NO:5, SEQ ID NO:7, SEQ ID NO:9, SEQ ID NO:10, or SEQ ID NO:11, or the complements of such polynucleotide sequences. A μUtr polynucleotide also can include a polynucleotide having a sequence identity of at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a reference μUtr polynucleotide.

As used herein, “sequence identity” refers to the identity between two polynucleotide sequences. Sequence identity is generally determined by aligning the residues of the two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate sequence is the sequence being compared to a known sequence. For example, two polynucleotide sequences can be compared using the Blastn program of the BLAST 2 search algorithm, as described by Tatiana et al., 1999 FEMS Microbiol Lett. 174:247-250, and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on.

In another aspect, this disclosure describes polynucleotide fragments. A polynucleotide fragment is a portion of an isolated μUtr polynucleotide as described herein. Such a portion may be several hundred nucleotides in length, for example about 100, about 200, about 300, about 400, about 500, about 600, about 700, about 800, about 900 or about 1000 nucleotides in length.

A polynucleotide as described herein may be formulated in a composition along with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with a μUtr polynucleotide without causing any undesirable biological effects or interacting in a deleterious manner with any of the other components of the pharmaceutical composition in which it is contained.

In embodiments in which a μUtr polypeptide is formulated in a pharmaceutical composition that includes a pharmaceutically acceptable carrier, the polynucleotide may be formulated and administered by methods known to those skilled in the art for delivering therapeutic polynucleotides.

This disclosure further describes a general method for efficiently purifying a polypeptide of interest. Generally, the method includes constructing a fusion polypeptide that includes a positively-charged tag and a second region that includes a functional portion of a polypeptide of interest. One can express the fusion polypeptide from a suitable host organism, collect the fusion polypeptide using routine methods appropriate for collecting polypeptides expressed by the host organism, and subject a sample that includes the collected fusion protein to cation exchange chromatography.

The positively-charged tag can be any an amino acid addition having at least 12 amino acids and a net positive charge. In certain embodiments, such a positively-charged tag can have at least one non-arginine amino acid residue. In other embodiments, the positively-charged tag can be positioned at the N-terminal end of the μUtr polypeptide. As discussed above, exemplary positively-charged tags include a TAT sequence (e.g., YGRKKRRQRRR, shown in amino acids 11-21 of SEQ ID NO:3, amino acids 11-21 of SEQ ID NO:5, amino acids 3-13 of SEQ ID NO:7, amino acids 3-16 of SEQ ID NO:9, amino acids 2-12 of SEQ ID NO:10, and SEQ ID NO:11) or a HA sequence (e.g., YPYDVPDYA, shown in amino acids 29-37 of SEQ ID NO:3, amino acids 29-37 of SEQ ID NO:5, amino acids 21-29 of SEQ ID NO:7, and amino acids 20-28 of SEQ ID NO:10).

As noted above with respect to a TAT-μUtr polypeptide, the positively-charged tag—e.g., a TAT sequence—can cause a polypeptide that otherwise would not bind to a cation exchange column to do so, thereby permitting scalable, cost-effective purification of the tag-containing fusion polypeptide. For example, the highly basic nature of the TAT protein transduction domain also can function as a tag that can facilitate binding of a polypeptide that contains the TAT protein transduction domain to a cation exchange column. Thus, a fusion polypeptide that includes, for example, a TAT tag may be isolatable in a scalable, single-step process using cation exchange chromatography. Moreover, the TAT region can provide cell transduction activity in the event that the purified TAT-containing polypeptide is desired for a use that is facilitated by having the TAT-containing polypeptide cross a biological membrane.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### FLAG-TAT-μUtr and FLAG-less TAT-μUtr Constructs

Generation of Human TAT-μUtrophin Baculovirus Constructs

Total RNA was isolated from HEK 293 cells using TRIzol® Reagent (Invitrogen; Carlsbad, Calif.). RT-PCR (SuperScript® One-Step RT-PCR kit with Platinum® Taq, Invitrogen; Carlsbad, Calif.) was performed on total RNA using primers to amplify the start methionine through spectrin-like repeat 3 and hinge 1 of the human utrophin transcript. Kozak consensus, TAT and HA encoding sequences were added to the resulting 2100 bp product using PCR with overlapping primers. Spectrin-like repeat 22 through the C-terminal stop codon of human utrophin were PCR amplified from a human utrophin cDNA-containing plasmid (kindly provided by Dr. Kay Davies). A recombinant PCR strategy was used to join the TAT-N-terminus through hinge 1 PCR fragment to the spectrin repeat 22 through stop codon fragment. The resulting 4700 bp TAT-μ-utrophin PCR product was cloned into the pCR®-Blunt vector (Invitrogen; Carlsbad, Calif.), digested with SpeI and XhoI, and subcloned into the pFastBac™1 vector (Invitrogen; Carlsbad, Calif.). Sequence verification revealed three mutations that were then corrected using site-directed mutatgenesis. The TAT-HA-N-terminus through hinge 1 PCR fragment was cloned into the pCR®Blunt vector, digested with SpeI and BplI, and subcloned into the corrected TAT-μ-utrophin pFastBac™1 vector to generate TAT-HA-μ-utrophin pFastBac™1 plasmid. Following extensive sequence verification of both human μ-utrophin constructs, DH10Bac™ cells (Invitrogen; Carlsbad, Calif.) were transformed with TAT-μ-utrophin pFastBac™1 plasmid or TAT-HA-μ-utrophin pFastBac™1 plasmid to generate recombinant bacmids. Sf9 insect cells were transfected with the bacmids to generate recombinant baculoviruses used for subsequent expression of human TAT-μ-utrophin or human TAT-HA-μ-utrophin. All PCR reactions were performed using PfuUltra™ or PfuUltra™ II Fusion HS high fidelity DNA polymerases (Stratagene; Cedar Creek, Tex.).

The FLAG-less TAT-HA-μUtr polypeptide is depicted in SEQ ID NO:10. The FLAG-less TAT-μUtr polypeptide is depicted in SEQ ID NO:11.

Generation of FLAG-TAT-μUtrophin

FLAG-tagged TAT-μ-utrophin was generated as previously described (Sonnemann et al., 2009 PLoS Med 6:e1000083).

Purification of FLAG-TAT-μUtr and FLAG-μUtr

FLAG-TAT-μUtr and FLAG-μUtr were purified by anti-FLAG M2 affinity chromatography as detailed previously (Sonnemann et al., 2009 PLoS Med 6:e1000083). For purification of TAT-μUtr, frozen infected Sf9 cell pellets were ground with a liquid nitrogen-cooled mortar and pestle and solubilized for 1 hour at 4° C. in 1% Triton X-100, 0.3 M NaCl in PBS, pH 7.5, and a cocktail of protease inhibitors (Sonnemann et al., 2009 PLoS Med 6:e1000083). The soluble supernatant obtained after 10 minutes centrifugation at 14,000×g was loaded onto a 20-ml SP Sepharose (Sigma-Aldrich; St. Louis, Mo.) column that was pre-equilibrated with 0.3 M NaCl in PBS, pH 7.5. The column was washed with 0.4 M NaCl in PBS, pH 7.5, and bound protein was eluted with 0.7 M NaCl in PBS, pH 7.5. Fractions containing TAT-μUtr were pooled, dialyzed 2×100 volumes of PBS, pH 7.5 at 4° C. Purified proteins were sterilized for injection by passage through a 0.22 μm filter and injected into the intraperitoneal cavity of mdx mice at a concentration of 1.5-3.0 mg/ml.

### Example 2

### Protein Labeling and Infrared Imaging

Fluorescent labeling of FLAG-TAT-μUtr and TAT-μUtr was performed as previously described (Sonnemann et al., 2009 PLoS Med 6:e1000083). Purified FLAG-TAT-μUtr or TAT-μUtr were diluted to 1.0 mg/ml in PBS and labeled with IRDye 800CW-High MW Protein Labeling Kit (LI-COR Biosciences; Lincoln, Nebr.) according to the manufacturer's instructions. The labeled proteins were sterilized with a 0.22 μm syringe filter prior to injection.

C57Bl/10ScSn-Dmd^{mdx}/J (The Jackson Laboratory; Bar Harbor, Me.) mice (n=2 per time point) were singly injected with either FLAG-TAT-μUtr or TAT-μUtr. At 3, 24, 48, and 72 hours post-injection, mice were euthanized and scanned for whole-body fluorescence using the Pearl® Imager (LI-COR Biosciences; Lincoln, Nebr.), using both the 800 nm (labeled protein) and 700 nm (background) channels. Quadriceps muscles and liver tissues were also frozen in liquid nitrogen, and later analyzed for tissue fluorescence. For analysis of SDS-extracts, frozen tissue was pulverized and protein was extracted as previously described (Mendell et al., 2010 N Engl J Med 363:1429-37). Lysates were separated by size by SDS-PAGE, transferred to nitrocellulose, and the membranes were scanned using the Odyssey® Infrared Imaging System (LI-COR Biosciences; Lincoln, Nebr.). Lysate fluorescence was normalized to protein load by densitometry of the Coomassie blue-stained gels after transfer.

### Example 3

### Treatment of Mdx Mice with TAT-μUtr

For all studies, protein (1.5 to 3.0 mg/ml) was administered by intraperitoneal injection at a dosage of 8.5 μg/g body mass while controls received an equal volume of sterile PBS. Short-term efficacy of TAT-μUtr was assessed as previously described (Sonnemann et al., 2009 PLoS Med 6:e1000083). Mice received twice-weekly injections of PBS (n=5) or TAT-μUtr (n=7) for 3 weeks starting at 18 days of age. Littermates were treated in parallel and unbiased of gender.

Long-term efficacy of TAT-μUtr was assessed with the same analyses as the short-term study. Mice received twice-weekly injections of PBS (n=8) or TAT-μUtr (n=7) for 13.5 weeks starting at 18 days of age. Animals were housed and treated in accordance with the standards set by the Institutional Animal Care and Use Committee at the University of Minnesota.

Grip Strength

Forelimb grip strength was tested with the Grip Strength Meter (Columbus Instruments; Columbus, Ohio) in all mice at the end of each study. Each mouse was held by the tail and lowered towards a triangular-shaped bar that was connected to a force transducer. After establishing a firm grip with the forepaws, mice were pulled by the tail in a direction parallel to the ground until they released the bar. Each mouse performed five serial pulls. The five pulls were averaged together for each mouse and normalized to body mass.

Protein Extracts

For samples enriched in membrane glycoproteins using WGA affinity chromatography (Sonnemann et al., 2009 PLoS Med 6:e1000083), pulverized muscle was solubilized 1:10 (w:v) in 5% digitonin solubilization buffer for one hour at 4° C. The solubilate was spun down at 1000 rpm for 10 minutes and the supernatant then loaded onto equilibrated WGA beads (50 μl beads per 1 ml supernatant, Vector Labs; Burlingame, Calif.) and mixed end-over-end overnight at 4° C. Beads were then pelleted and washed three times in 10% digitonin wash buffer before protein was eluted in 0.3 M NAG elution buffer.

Electrophoresis/Western Blotting

Conventional 3-12% gradient gels were used to detect sarcolemmal proteins via SDS-PAGE. All western blotting was performed as described (Mendell et al., 2010 N Engl J Med 363:1429-37) using the following primary antibodies: anti-utrophin mAb 8A4 (1:50; Santa Cruz Biotechnology®, Inc.; Santa Cruz, Calif.), anti-FLAG pAb (1:1000; Sigma-Aldrich, St. Louis, Mo.), anti-syntrophin mAb 1351 (1:1000, Abcam®, Cambridge, Mass.), anti-α-sarcoglycan mAb NCL-a-Sarc (1:50; Novocastra Reagents (a division of Leica Microsystems Inc.); Buffalo Grove, Ill.), anti-β-dystroglycan mAb NCL-b-DG (1:50; Novocastra Reagents (a division of Leica Microsystems Inc.); Buffalo Grove, Ill.), and anti-dihydropyridine receptor mAb IIC12D4 (1:500, Developmental Studies Hybridoma Bank; Iowa City, Iowa). Secondary antibodies were diluted (1:5000) and detected with the ODYSSEY Infrared Imaging System (LI-COR Biosciences; Lincoln, Nebr.) using the 700 and 800 nm channels.

Histological and Morphometric Analysis

Individual muscles were dissected, coated with OCT (TissueTek® (a division of Sakura); Torrance, Calif.), and rapidly frozen in liquid nitrogen-cooled isopentane. Cryosections of 10 μm thickness were cut on a Leica CM3050 cryostat and stained with hematoxylin and eosin-phloxine. Images were collected on a Zeiss Axiovert 25 microscope and compiled into montages of entire sections in ImagePro Plus and exported to Scion Image for morphometric analyses. The percentage of centrally nucleated fibers was determined from one muscle of each mouse with every fiber scored for analysis.

Immunofluorescence

Cryosections of 10 μm thickness were stained with primary antibodies as described (Wehling et al., 2001 J Cell Biol 155:123-31). Stacks of images were obtained on a DeltaVision personalDV deconvolution microscopy system using a 40× oil objective. The image stacks were deconvolved and projections imported as TIF files into CorelDraw X4 for figure preparation. Primary monoclonal antibodies used were identical to those described for western blotting above, with the addition of anti-laminin mAb 4H8-2 (Sigma-Aldrich; St. Louis, Mo.), anti-dystrobrevin (Novocastra Reagents (a division of Leica Microsystems Inc.); Buffalo Grove, Ill.), and anti-nNOS pAb Z-RNN3 (Invitrogen; Carlsbad, Calif.).

EDL Contractile Properties

Contractile properties of the EDL muscle were measured as previously described (Sonnemann et al., 2009 PLoS Med 6:e1000083). Briefly, mice were anesthetized with sodium pentobarbital (100 mg/kg body mass). EDL muscles were dissected and mounted to a dual-mode muscle lever system (300B-LR; Aurora Scientific Inc.; Aurora, ON, Canada) in a 0.38-ml bath assembly filled with Krebs-Ringer bicarbonate buffer that was maintained at 25° C. and perfused with 95% O₂. Maximal isometric tetanic force (Pₒ) was determined by stimulating muscles for 400 ms at 180 Hz and 150 V (Grass S48 stimulator delivered through a SIU5D stimulus isolation unit; Grass Telefactor; Warwick, R.I.). Specific force was calculated by normalizing Pₒ to muscle cross-sectional area: muscle weight divided by the product of muscle density (1.06 g/ml) and fiber length. The eccentric injury protocol consisted of 5 eccentric contractions, passively shortening the muscle from Lₒ to 0.95 Lₒ over 3 s, stimulating tetanically for 200 ms as the muscle lengthened to 1.05 Lₒ at 0.5 Lₒ/s, and then passively returning to Lₒ. Each eccentric contraction was separated by 3 minutes of rest. Contractility and injury protocols were performed on EDL muscles from both the right and left legs, and they were averaged together as a single data point for each mouse. The investigator (KAB) was blinded to the treatment of each mdx mouse.

Serum Creatine Kinase Analysis

Retro-orbital bleeds were performed on anesthetized mice as described (Wehling et al., 2001 J Cell Biol 155:123-31). Data were collected in U/mL.

Statistical Analysis

Data are reported as means±SEM. Short-term treatments were analyzed with Student's t-tests comparing mice treated with TAT-μUtr and PBS. For the long-term study, Student's t-tests were performed between FLAG-TAT-μUtr and TAT-μUtr groups, and no significant differences existed for all tested variables (P≧0.130). These groups were pooled and compared against PBS-treated mice. Significance was set at P<0.05.

### EMBODIMENTS

### Embodiment 1

An isolated polypeptide comprising:

a μ-utrophin region or an anti-dystrophinopathic fragment thereof operationally linked to a second region effective to transduce the fusion protein into mammalian muscle cells;

with the proviso that the isolated polypeptide does not include SEQ ID NO:1.

### Embodiment 2

The isolated polypeptide of Embodiment 1 wherein the μ-utrophin region or an anti-dystrophinopathic fragment thereof comprises a deletion of at least one spectrin-like repeat compared to native utrophin.

### Embodiment 3

The isolated polypeptide of Embodiment 1 or Embodiment 2 wherein the second region comprises amino acids 3-13 of SEQ ID NO:7.

### Embodiment 4

The isolated polypeptide of any one of Embodiments 1-3 wherein the second region comprises amino acids 21-29 of SEQ ID NO:7.

### Embodiment 5

A composition comprising:

an isolated polypeptide of any one of Embodiments 1-4, or a pharmaceutically suitable salt thereof, in combination with a pharmaceutically acceptable carrier.

### Embodiment 6

An isolated nucleic acid expression construct encoding a polypeptide, the nucleic acid expression construct comprising:

a first nucleic acid region that encodes a μ-utrophin polypeptide or an anti-dystrophinopathic fragment thereof;

a second nucleic acid region that encodes an amino acid sequence effective to transduce the μ-utrophin polypeptide into mammalian muscle cells operationally linked to the first nucleic acid region;

with the proviso that the polypeptide does not include SEQ ID NO:1.

### Embodiment 7

The isolated polynucleotide of Embodiment 6 wherein the μ-utrophin polypeptide or an anti-dystrophinopathic fragment thereof comprises a deletion of at least one spectrin-like repeat compared to native utrophin.

### Embodiment 8

The isolated polynucleotide of Embodiment 6 or Embodiment 7 wherein the second nucleic acid region encodes a polypeptide that comprises amino acids 3-13 of SEQ ID NO:7.

### Embodiment 9

The isolated polynucleotide of any one of Embodiments 6-8 wherein the second nucleic acid region encodes a polypeptide that comprises amino acids 21-29 of SEQ ID NO:7.

### Embodiment 10

A method of treating a dystrophinopathy in a subject, the method comprising:

administering to a subject in need such treatment an anti-dystrophinopathic amount of an isolated polypeptide of any one of Embodiments 1-4.

### Embodiment 11

The method of Embodiment 10 wherein the dystrophinopathy comprises Duchenne muscular dystrophy.

### Embodiment 12

The method of Embodiment 10 or Embodiment 11 wherein the isolated polypeptide is administered at least twice per week.

### Embodiment 13

The method of any one of Embodiments 10-12 wherein the isolated polypeptide is administered for at least 13 weeks.

### Embodiment 14

A method of isolating the polypeptide of any one of Embodiments 1-4, the method comprising:

receiving a sample comprising the polypeptide;

performing cation exchange chromatography on at least a portion of the sample; and

recovering the polypeptide at a purity of at least 86%.

### Embodiment 15

The method of Embodiment 14 wherein the polypeptide is recovered with a yield of at least 90%.

### Embodiment 16

A method of isolating the polypeptide of any one of Embodiments 1-4, the method comprising:

receiving a sample comprising the polypeptide;

performing cation exchange chromatography on at least a portion of the sample; and

recovering the polypeptide at a yield of at least 90%.

### Embodiment 17

A method of isolating a polypeptide that comprises a net negative charge, the method comprising:

obtaining a sample comprising a fusion polypeptide comprising:
the polypeptide comprising a net negative charge, and
a positively charged tag comprising at least 12 amino acids,
wherein at least one of the following is true:
   the positively charged tag comprises at least one non-arginine amino acid residue, or
   the positively charged tag is located at the N-terminal of the fusion polypeptide; and

performing cation exchange chromatography on at least a portion of the sample.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO:1
DYKDDDDK
SEQ ID NO: 2
(Flag-TAT-HA Utrophin ΔR7-22)
atggactacaaggacgacgatgacaagggctacggccgcaagaaacgccgccagcgccgccgcggtggatccaccatgtccggctatccatatgacgtcccag
actatgctggctccatggccaagtatggggaccttgaagccaggcctgatgatgggcagaacgaattcagtgacatcattaagtccagatctgatgaacacaa
tgatgtacagaagaaaaccataccaaatggataaacgctcgattttccaagagtgggaaaccacccatcagtgatatgttctcagacctcaaagatgggagaa
agctcttggatcttctcgaaggcctcacaggaacatcattgccaaaggaacgtggttccacaagggtgcatgccttaaacaatgtcaaccgagtgctacaggt
tttacatcagaacaatgtggacttggtgaatattggaggcacggacattgtggatggaaatcccaagctgactttagggttactctggagcatcattctgcac
tggcaggtgaaggatgtcatgaaagatatcatgtcagacctgcagcagacaaacagcgagaagatcctgctgagctgggtgcggcagaccaccaggccctaca
gtcaagtcaacgtcctcaacttcaccaccagctggaccgatggactcgcgttcaacgccgtgctccaccggcacaaaccagatctcttcagctgggacagagt
ggtcaaaatgtccccaattgagagacttgaacatgcttttagcaaggcccacacttatttgggaattgaaaagcttctagatcctgaagatgttgctgtgcat
ctccctgacaagaaatccataattatgtatttaacgtctctgtttgaggtgatcctcagcaagtcacgatagatgccatccgagaggtggagactctcccaag
gaagtataagaaagaatgtgaagaggaagaaattcatatccagagtgcagtgctggcagaggaaggccagagtccccgagctgagacccctagcaccgtcact
gaagtggacatggatttggacagctaccagatagcgctagaggaagtgctgacgtggctgctgtccgcggaggacacgttccaggagcaagatgacatttctg
atgatgtcgaagaagtcaaagagcagtttgctacccatgaaacttttatgatggagctgacagcacaccagagcagcgtggggagcgtcctgcaggctggcaa
ccagctgatgacacaagggactctgtcagaggaggaggagtttgagatccaggaacagatgaccttgctgaatgcaaggtgggaggcgctccgggtggagagc
atggagaggcagtcccggctgcacgacgctctgatggagctgcagaagaaacagctgcagcagctctcaagctggctggccctcacagaagagcgcattcaga
agatggagagcctcccgctgggtgatgacctgccctccctgcagaagctgcttcaagaacataaaagtttgcaaaatgaccttgaagctgaacaggtgaaggt
aaattccttaactcacatggtggtgattgtggatgaaaacagtggggagagtgccacagctcttctggaagatcagttacagaaactgggtgagcgctggaca
gctgtatgccgctggactgaagaacgttggaacaggttgcaagaaatcagtattctgtggcaggaattattggaagagcagtgtctgttggaggcttggctca
ccgaaaaggaagaggctttgaataaagttcaaaccagcaactttaaagaccagaaggaactaagtgtcagtgtccggcgtctggctatattgaaggaagacat
ggaaatgaagaggcagactctggatcaactgagtgagattggccaggatgtgggccaattactcagtaatcccaaggcatctaagaagatgaacagtgactct
gaggagctaacacagagatgggattctctggttcagagactcgaagactcttctaaccaggtgactcaggcggtagcgaagctcggcatgtcccagattccac
agaaggacctattggagaccgttcatgtgagagaacaagggatggtgaagaagcccaagcaggaactgcctcctcctcccccaccaaagaagagacagattca
cgtggacgtggaggccaagaaaaagtttgatgctataagtacagagctgctgaactggattttgaaatcaaagactgccattcagaacacagagatgaaagaa
tataagaagtcgcaggagacctcaggaatgaaaaagaaattgaagggattagagaaagaacagaaggaaaatctgccccgactggacgaactgaatcaaaccg
gacaaaccctccgggagcaaatgggaaaagaaggcctttccactgaagaagtaaacgatgttctggaaagggtttcgttggagtggaagatgatatctcagca
gctagaagatctgggaaggaagatccagctgcaggaagatataaatgcttattttaagcagcttgatgccattgaggagaccatcaaggagaaggaagagtgg
ctgaggggcacacccatttctgaatcgccccggcagcccttgccaggcttaaaggattcttgccagagggaactgacagatctccttggccttcaccccagaa
ttgagacgctgtgtgcaagctgttcagccctgaagtctcagccctgtgtcccaggttttgtccagcagggttttgacgaccttcgacatcattaccaggctgt
gcggaaggctttagaggaataccaacaacaactagaaaatgagctgaagagccagcctggacccgcgtatttggacacactgaataccctgaaaaaaatgcta
agcgagtcagaaaaggcggcccaggcctctctgaatgccctgaacgatcccatagcggtggagcaggccctgcaggagaaaaaggcccttgatgaaacccttg
agaatcagaaacatacgttacataagctttcagaagaaacgaagactttggagaaaaatatgcttcctgatgtggggaaaatgtataaacaagaatttgatga
tgtccaaggcagatggaataaagtaaagaccaaggtttccagagacttacacttgctcgaggaaatcgcccacagagattttgggccatcttctcaacacttt
ctgtccacttcagtccagctgccgtggcagagatccatttcacataataaagtgccctattacatcaaccatcaaacacagacaacctgttgggatcatccta
aaatgactgagctcttccaatcccttgctgatctgaataatgtacgtttctctgcctaccgcacagcaatcaaaattcgaaggctgcaaaaagcattatgtct
ggatctcttagagctgaatacgacgaatgaagttttcaagcagcacaaactgaaccaaaatgatcagctcctgagtgtcccagacgtcatcaactgtctgacc
accacttacgatgggcttgagcagctgcacaaggacttggtcaatgttccactctgcgtcgatatgtgtctcaactggctgctcaacgtatacgacacgggcc
ggactggaaaaattcgggtacagagtctgaagattggattgatgtctctctccaaaggcctcttagaagagaaatacagatgtctctttaaggaggtggcagg
gccaacagagatgtgtgaccagcggcagcttggcctgctacttcacgatgccatccagatccctaggcagctgggggaagtagcagcctttgggggcagtaac
attgagcccagtgtccgcagctgcttccagcagaataacaacaagccagaaatcagtgtgaaggagtttatagactggatgcatttggaaccccagtccatgg
tgtggttgccggttctgcatcgggtcgcagctgctgagactgcaaaacatcaggccaaatgcaacatctgcaaagaatgcccgattgttgggttcagatacag
gagcctaaagcattttaattatgatgtctgccagagttgcttcttttctggaagaacagcaaagggccacaagttacattacccgatggtagaatactgcata
ccgacaacatctggggaagatgtgagagatttcactaaggtgctgaagaacaagttcaggtccaagaaatattttgccaaacatcctcggcttggctacctgc
ctgtccagaccgtgctggaaggggacaacttagaaactcctatcacgctcatcagtatgtggccagagcactatgacccctcccagtcccctcagctgtttca
tgatgacacccactcaagaatagagcaatacgctacacgactggcccagatggaaaggacaaacgggtccttcctaactgatagcagctctacaacaggaagc
gtggaggatgagcatgccctcatccagcagtactgccagaccctgggcggggagtcacctgtgagtcagccgcagagtccagctcagatcctgaagtccgtgg
agagggaagagcgtggggaactggagcggatcattgctgacttggaggaagagcaaagaaatctgcaggtggagtatgagcagctgaaggagcagcacctaag
aaggggtctccctgtgggctcccctccagactccatcgtatctcctcaccacacatctgaggactcagaacttatagcagaagctaaactcctgcggcagcac
aaagggcggctggaggcgaggatgcaaattttggaagatcacaataaacagctggagtctcagctgcaccgcctcagacagctcctggagcagcctgactctg
actcccgcatcaatggtgtctccccctgggcttccccacagcattctgcattgagctactcacttgacactgacccaggcccacagttccaccaggcagcatc
tgaggacctgctggccccacctcacgacactagcacggacctcacggacgtgatggagcagatcaacagcacgtttccctcttgcagctcaaatgtccccagc
aggccacaggcaatgtga
SEQ ID NO: 3
(FLAG-tagged TAT-HA Utrophin ΔR7-22)
MDYKDDDDKGYGRKKRRORRRGGSTMSGYPYDVPDYAGSMA [symbol] GDLEARPDDGQNEFSDIIKSRSD
EHNDVQKKTFTKWINARFSKSGKPPISDMFSDLKDGRKLLDLLEGLTGTSLPKERGSTRVHALNNVNR
VLQVLHQNNVDLVNIGGTDIVDGNPKLTLGLLWSIILHWQVKDVMKDIMSDLQQTNSEKILLSWVRQT
TRPYSQVNVLNFTTSWTDGLAFNAVLHRHKPDLFSWDRVVKMSPIERLEHAFSKAHTYLGIEKLLDPE
DVAVHLPDKKSIIMYLTSLFEVLPQQVTIDAIREVETLPRKYKKECEEEEIHIQSAVLAEEGQSPRAETPST
VTEVDMDLDSYQIALEEVLTWLLSAEDTFQEQDDISDDVEEVKEQFATHETFMMELTAHQSSVGSVLQ
AGNQLMTQGTLSEEEEFEIQEQMTLLNARWEALRVESMERQSRLHDALMELQKKQLQQLSSWLALTE
ERIQKMESLPLGDDLPSLQKLLQEHKSLQNDLEAEQVKVNSLTHMVVIVDENSGESATALLEDQLQKL
GERWTAVCRWTEERWNRLQEISILWQELLEEQCLLEAWLTEKEEALNKVQTSNFKDQKELSVSVRRLA
ILKEDMEMKRQTLDQLSEIGQDVGQLLSNPKASKKMNSDSEELTQRWDSLVQRLEDSSNQVTQAVAK
LGMSQIPQKDLLETVHVREQGMVKKPKQELPPPPPPKKRQIHVDVEAKKKFDAISTELLNWILKSKTAI
QNTEMKEYKKSQETSGMKKKLKGLEKEQKENLPRLDELNQTGQTLREQMGKEGLSTEEVNDVLERVS
LEWKMISQQLEDLGRKIQLQEDINAYFKQLDAIEETIKEKEEWLRGTPISESPRQPLPGLKDSCQRELTDL
LGLHPRIETLCASCSALKSQPCVPGFVQQGFDDLRHHYQAVRKALEEYQQQLENELKSQPGPAYLDTL
NTLKKMLSESEKAAQASLNALNDPIAVEQALQEKKALDETLENQKHTLHKLSEETKTLEKNMLPDVGK
MYKQEFDDVQGRWNKVKTKVSRDLHLLEEIAHRDFGPSSQHFLSTSVQLPWQRSISHNKVPYYTNHQT
QTTCWDHPKMTELFQSLADLNNVRFSAYRTAIKIRRLQKALCLDLLELNTTNEVFKQHKLNQNDQLLS
VPDVINCLTTTYDGLEQLHKDLVNVPLCVDMCLNWLLNVYDTGRTGKIRVQSLKIGLMSLSKGLLEEK
YRCLEKEVAGPTEMCDQRQLGLLLHDAIQIPRQLGEVAAFGGSNIEPSVRSCFQQNNNKPEISVKEFIDW
MHLEPQSMVWLPVLHRVAAAETAKHQAKCNICKECPIVGFRYRSLKHFNYDVCQSCFFSGRTAKGHK
LHYPMVEYCIPTTSGEDVRDFTKVLKNKFRSKKYFAKHPRLGYLPVQTVLEGDNLETPITLISMWPEHY
DPSQSPQLFHDDTHSRIEQYATRLAQMERTNGSFLTDSSSTTGSVEDEHALIQQYCQTLGGESPVSQPQS
PAQILKSVEREERGELERIIADLEEEQRNLQVEYEQLKEQHLRRGLPVGSPPDSIVSPHHTSEDSELIAEAK
LLRQHKGRLEARMQILEDHNKQLESQLHRLRQLLEQPDSDSRINGVSPWASPQHSALSYSLDTDPGPQF
HQAASEDLLAPPHDTSTDLTDVMEQINSTFPSCSSNVPSRPQAM
SEQ ID NO: 3 Notes:
Flag tag (bold)
TAT PTD (bold underlined)
HA tag (underlined)
First three amino acids of utrophin (bold underlined italics)
SEQ ID NO: 4
(Flag-TAT-HA Utrophin ΔR11-22)
atggactacaaggacgacgatgacaagggctacggccgcaagaaacgccgccagcgccgccgcggtggatccaccatgtccggctatccatatgacgtcccag
actatgctggctccatggccaagtatggggaccttgaagccaggcctgatgatgggcagaacgaattcagtgacatcattaagtccagatctgatgaacacaa
tgatgtacagaagaaaacctttaccaaatggataaacgctcgattttccaagagtgggaaaccacccatcagtgatatgttctcagacctcaaagatgggaga
aagctcttggatcttctcgaaggcctcacaggaacatcattgccaaaggaacgtggttccacaagggtgcatgccttaaacaatgtcaaccgagtgctacagg
ttttacatcagaacaatgtggacttggtgaatattggaggcacggacattgtggatggaaatcccaagctgactttagggttactctggagcatcattctgca
ctggcaggtgaaggatgtcatgaaagatatcatgtcagacctgcagcagacaaacagcgagaagatcctgctgagctgggtgcggcagaccaccaggccctac
agtcaagtcaacgtcctcaacttcaccaccagctggaccgatggactcgcgttcaacgccgtgctccaccggcacaaaccagatctcttcagctgggacagag
tggtcaaaatgtccccaattgagagacttgaacatgctatagcaaggcccacacttatttgggaattgaaaagcttctagatcctgaagatgttgctgtgcat
ctccctgacaagaaatccataattatgtatttaacgtctctgtttgaggtgcttcctcagcaagtcacgatagatgccatccgagaggtggagactctcccaa
ggaagtataagaaagaatgtgaagaggaagaaattcatatccagagtgcagtgctggcagaggaaggccagagtccccgagctgagacccctagcaccgtcac
tgaagtggacatggataggacagctaccagatagcgctagaggaagtgctgacgtggctgctgtccgcggaggacacgttccaggagcaagatgacatttctg
atgatgtcgaagaagtcaaagagcagtttgctacccatgaaacttttatgatggagctgacagcacaccagagcagcgtggggagcgtcctgcaggctggcaa
ccagctgatgacacaagggactctgtcagaggaggaggagtttgagatccaggaacagatgaccttgctgaatgcaaggtgggaggcgaccgggtggagagca
tggagaggcagtcccggctgcacgacgctctgatggagctgcagaagaaacagagcagcagctacaagctggctggccctcacagaagagcgcattcagaaga
tggagagcctcccgctgggtgatgacctgccctccctgcagaagctgatcaagaacataaaagtttgcaaaatgaccttgaagctgaacaggtgaaggtaaat
tccttaactcacatggtggtgattgtggatgaaaacagtggggagagtgccacagctatctggaagatcagttacagaaactgggtgagcgctggacagctgt
atgccgctggactgaagaacgttggaacaggttgcaagaaatcagtattctgtggcaggaattattggaagagcagtgtctgttggaggcttggctcaccgaa
aaggaagaggctttgaataaagttcaaaccagcaactttaaagaccagaaggaactaagtgtcagtgtccggcgtctggctatattgaaggaagacatggaaa
tgaagaggcagactctggatcaactgagtgagattggccaggatgtgggccaattactcagtaatcccaaggcatctaagaagatgaacagtgactctgagga
gctaacacagagatgggattctctggttcagagactcgaagactcttctaaccaggtgactcaggcggtagcgaagctcggcatgtcccagattccacagaag
gacctattggagaccgttcatgtgagagaacaagggatggtgaagaagcccaagcaggaactgcctcctcctcccccaccaaagaagagacagattcacgtgg
acgtggaggccaagaaaaagtttgatgctataagtacagagctgctgaactggattttgaaatcaaagactgccattcagaacacagagatgaaagaatataa
gaagtcgcaggagacctcaggaatgaaaaagaaattgaagggattagagaaagaacagaaggaaaatctgccccgactggacgaactgaatcaaaccggacaa
accctccgggagcaaatgggaaaagaaggcctttccactgaagaagtaaacgatgttctggaaagggtttcgttggagtggaagatgatatctcagcagctag
aagatctgggaaggaagatccagctgcaggaagatataaatgcttattttaagcagcttgatgccattgaggagaccatcaaggagaaggaagagtggctgag
gggcacacccatttctgaatcgccccggcagcccttgccaggcttaaaggattcttgccagagggaactgacagatctccttggccttcaccccagaattgag
acgctgtgtgcaagctgttcagccctgaagtctcagccctgtgtcccaggttttgtccagcagggttttgacgaccttcgacatcattaccaggctgtgcgga
aggctttagaggaataccaacaacaactagaaaatgagctgaagagccagcctggacccgcgtatttggacacactgaataccctgaaaaaaatgctaagcga
gtcagaaaaggcggcccaggcctctctgaatgccctgaacgatcccatagcggtggagcaggccctgcaggagaaaaaggcccttgatgaaacccttgagaat
cagaaacatacgttacataagctttcagaagaaacgaagactttggagaaaaatatgcttcctgatgtggggaaaatgtataaacaagaatttgatgatgtcc
aaggcagatggaataaagtaaagaccaaggtttccagagacttacacttgctcgaggaaatcacccccagactccgagattttgaggctgattcagaagtcat
tgagaagtgggtgagtggcatcaaagacttcctcatgaaagaacaggctgctcaaggagacgctgctgcgctgcagagccagcttgaccaatgtgctacgttt
gctaatgaaatcgaaaccatcgagtcatctctgaagaacatgagggaagtagagactagccttcagaggtgtccagtcactggagtcaagacatgggtacagg
caagactagtggattaccaatcccaactggagaaattcagcaaagagattgctattcaaaaaagcaggctgtcagatagtcaagaaaaagccctgaacttgaa
aaaggatttggctgagatgcaggagtggatggcacaggctgaagaggactacctggagagggacttcgagtacaaatctccagaaagaactcgagagtgcggt
ggaggaaatgaagagggcaaaagaggaggtgctgcagaaggaggtgagggtgaaaattctgaaggacagcatcaagctggtggctgccaaggtgccctctggt
ggccaggagttgacgtcggaattcaacgaggtgctggagagctaccagcttctgtgcaatagaattcgagggaagtgccacacactggaggaggtctggtctt
gctgggtggagctgcttcactatctggacctggagaccacgtggttgaacaccttggaggagcgcgtgaggagcacggaggccctgcctgagagggcagaagc
tgttcatgaagctctggagtctcttgagtctgttttgcgccatccggcggataatcgcacccagattcgggaacttgggcagactctgattgatggtggaatc
ctggatgacataatcagcgagaagctggaggcttttaacagccgctacgaagagctgagtcacttggcggagagcaaacagatttctttggagaagcaactcc
aggtcctccgcgaaactgaccacatgcttcaggtgctgaaggagagcctgggggagctggacaaacagcttaccacatacctgacggacaggatcgatgcctt
ccaactgccacaggaagctcagaagatccaagccgaaatctcagcccatgagctcaccctggaggagctgaggaagaatgtgcgctcccagcccccgacgtcc
cctgagggcagggccaccagaggaggaagtcagatggacatgctacagaggaaacttcgagaggtctccaccaaattccagcttgcccacagagattttgggc
catcttctcaacactttctgtccacttcagtccagctgccgtggcagagatccatttcacataataaagtgccctattacatcaaccatcaaacacagacaac
ctgttgggatcatcctaaaatgactgagctcttccaatcccttgctgatctgaataatgtacgtttctctgcctaccgcacagcaatcaaaattcgaaggctg
caaaaagcattatgtctggatctcttagagctgaatacgacgaatgaagttttcaagcagcacaaactgaaccaaaatgatcagctcctgagtgtcccagacg
tcatcaactgtctgaccaccacttacgatgggcttgagcagctgcacaaggacttggtcaatgttccactctgcgtcgatatgtgtctcaactggctgctcaa
cgtatacgacacgggccggactggaaaaattcgggtacagagtctgaagattggattgatgtctctctccaaaggcctcttagaagagaaatacagatgtctc
tttaaggaggtggcagggccaacagagatgtgtgaccagcggcagcttggcctgctacttcacgatgccatccagatccctaggcagctgggggaagtagcag
cctttgggggcagtaacattgagcccagtgtccgcagctgcttccagcagaataacaacaagccagaaatcagtgtgaaggagtttatagactggatgcattt
ggaaccccagtccatggtgtggttgccggttctgcatcgggtcgcagctgctgagactgcaaaacatcaggccaaatgcaacatctgcaaagaatgcccgatt
gttgggttcagatacaggagcctaaagcattttaattatgatgtctgccagagttgcttcttttctggaagaacagcaaagggccacaagttacattacccga
tggtagaatactgcataccgacaacatctggggaagatgtgagagatttcactaaggtgctgaagaacaagttcaggtccaagaaatattttgccaaacatcc
tcggcttggctacctgcctgtccagaccgtgctggaaggggacaacttagaaactcctatcacgctcatcagtatgtggccagagcactatgacccctcccag
tcccctcagctgtttcatgatgacacccactcaagaatagagcaatacgctacacgactggcccagatggaaaggacaaacgggtccttcctaactgatagca
gctctacaacaggaagcgtggaggatgagcatgccctcatccagcagtactgccagaccctgggcggggagtcacctgtgagtcagccgcagagtccagctca
gatcctgaagtccgtggagagggaagagcgtggggaactggagcggatcattgctgacttggaggaagagcaaagaaatctgcaggtggagtatgagcagctg
aaggagcagcacctaagaaggggtctccctgtgggctcccctccagactccatcgtatctcctcaccacacatctgaggactcagaacttatagcagaagcta
aactcctgcggcagcacaaagggcggctggaggcgaggatgcaaattttggaagatcacaataaacagctggagtctcagctgcaccgcctcagacagctcct
ggagcagcctgactctgactcccgcatcaatggtgtctccccctgggcttccccacagcattctgcattgagctactcacttgacactgacccaggcccacag
ttccaccaggcagcatctgaggacctgctggccccacctcacgacactagcacggacctcacggacgtgatggagcagatcaacagcacgtttccctcttgca
gctcaaatgtccccagcaggccacaggcaatgtga
SEQ ID NO: 5
(Flag-TAT-HA Utrophin ΔR11-22)
MDYKDDDDKGYGRKKRRORRRGGSTMSGYPYDVPDYAGSM [symbol] GDLEARPDDGQNEFSDIIKSRSD
EHNDVQKKTFTKWINARFSKSGKPPISDMFSDLKDGRKLLDLLEGLTGTSLPKERGSTRVHALNNVNR
VLQVLHQNNVDLVNIGGTDIVDGNPKLTLGLLWSIILHWQVKDVMKDIMSDLQQTNSEKILLSWVRQT
TRPYSQVNVLNFTTSWTDGLAFNAVLHRHKPDLFSWDRVVKMSPIERLEHAFSKAHTYLGIEKLLDPE
DVAVHLPDKKSIIMYLTSLFEVLPQQVTIDAIREVETLPRKYKKECEEEEIHIQSAVLAEEGQSPRAETPST
VTEVDMDLDSYQIALEEVLTWLLSAEDTFQEQDDISDDVEEVKEQFATHETFMMELTAHQSSVGSVLQ
AGNQLMTQGTLSEEEEFEIQEQMTLLNARWEALRVESMERQSRLHDALMELQKKQLQQLSSWLALTE
ERIQKMESLPLGDDLPSLQKLLQEHKSLQNDLEAEQVKVNSLTHMVVIVDENSGESATALLEDQLQKL
GERWTAVCRWTEERWNRLQEISILWQELLEEQCLLEAWLTEKEEALNKVQTSNFKDQKELSVSVRRLA
ILKEDMEMKRQTLDQLSEIGQDVGQLLSNPKASKKMNSDSEELTQRWDSLVQRLEDSSNQVTQAVAK
LGMSQIPQKDLLETVHVREQGMVKKPKQELPPPPPPKKRQIHVDVEAKKKFDAISTELLNWILKSKTAI
QNTEMKEYKKSQETSGMKKKLKGLEKEQKENLPRLDELNQTGQTLREQMGKEGLSTEEVNDVLERVS
LEWKMISQQLEDLGRKIQLQEDINAYFKQLDAIEETIKEKEEWLRGTPISESPRQPLPGLKDSCQRELTDL
LGLHPRIETLCASCSALKSQPCVPGFVQQGFDDLRHHYQAVRKALEEYQQQLENELKSQPGPAYLDTL
NTLKKMLSESEKAAQASLNALNDPIAVEQALQEKKALDETLENQKHTLHKLSEETKTLEKNMLPDVGK
MYKQEFDDVQGRWNKVKTKVSRDLHLLEEITPRLRDFEADSEVIEKWVSGIKDFLMKEQAAQGDAAA
LQSQLDQCATFANEIETIESSLKNMREVETSLQRCPVTGVKTWVQARLVDYQSQLEKFSKEIAIQKSRLS
DSQEKALNLKKDLAEMQEWMAQAEEDYLERDFEYKSPEELESAVEEMKRAKEEVLQKEVRVKTLKDS
IKLVAAKVPSGGQELTSEFNEVLESYQLLCNRIRGKCHTLEEVWSCWVELLHYLDLETTWLNTLEERVR
STEALPERAEAVHEALESLESVLRHPADNRTQIRELGQTLIDGGILDDIISEKLEAFNSRYEELSHLAESKQ
ISLEKQLQVLRETDHMLQVLKESLGELDKQLTTYLTDRIDAFQLPQEAQKTQAEISAHELTLEELRKNVR
SQPPTSPEGRATRGGSQMDMLQRKLREVSTKFQLAHRDFGPSSQHFLSTSVQLPWQRSISHNKVPYYIN
HQTQTTCWDHPKMTELFQSLADLNNVRFSAYRTAIKIRRLQKALCLDLLELNTTNEVFKQHKLNQNDQ
LLSVPDVINCLTTTYDGLEQLRKDLVNVPLCVDMCLNWLLNVYDTGRTGKIRVQSLKIGLMSLSKGLL
EEKYRCLFKEVAGPTEMCDQRQLGLLLHDAIQIPRQLGEVAAFGGSNIEPSVRSCFQQNNNKPEISVKEF
IDWMNLEPQSMVWLPVLHRVAAAETAKHQAKCNICKECPIVGFRYRSLKHFNYDVCQSCFFSGRTAK
GHKLHYPMVEYCIPTTSGEDVRDFTKVLKNKFRSKKYFAKHPRLGYLPVQTVLEGDNLETPITLISMWP
EHYDPSQSPQLFHDDTHSRIEQYATRLAQMERTNGSFLTDSSSTTGSVEDEHALIQQYCQTLGGESPVSQ
PQSPAQILKSVEREERGELERIIADLEEEQRNLQVEYEQLKEQHLRRGLPVGSPPDSIVSPHHTSEDSELIA
EAKLLRQHKGRLEARMQILEDHNKQLESQLHRLRQLLEQPDSDSRINGVSPWASPQHSALSYSLDTDPG
PQFHQAASEDLLAPPHDTSTDLTDVMEQTNSTFPSCSSNVPSRPQAM.
SEQ ID NO: 5 Notes:
Flag tag (bold)
TAT PTD (bold underlined)
HA tag (underlined)
First three amino acids of utrophin (bold underlined italics)
SEQ ID NO: 6
(TAT-HA Utrophin ΔR4-21)
atgggctacggccgcaagaaacgccgccagcgccgccgcggtggatccaccatgtccggctatccatatgacgtcccagactatgctggctccatggccaagtat
ggggaccttgaagccaggcctgatgatgggcagaacgaattcagtgacatcattaagtccagatctgatgaacacaatgatgtacagaagaaaacctttaccaaa
tggataaacgctcgattttccaagagtgggaaaccacccatcagtgatatgttctcagacctcaaagatgggagaaagctcttggatcttctcgaaggcctcaca
ggaacatcattgccaaaggaacgtggttccacaagggtgcatgccttaaacaatgtcaaccgagtgctacaggttttacatcagaacaatgtggacttggtgaat
attggaggcacggacattgtggatggaaatcccaagctgactttagggttactctggagcatcattctgcactggcaggtgaaggatgtcatgaaagatatcatg
gtcagacctgcacagacaaacagcgagaagatcctgctgagctgggtgcggcagaccaccaggccctacagtcaagtcaacgtcctcaacttcaccaccagctgg
accgatggactcgcgttcaacgccgtgctccaccggcacaaaccagatctcttcagctgggacagagtggtcaaaatgtccccaattgagagacttgaacatgct
tttagcaaggcccacacttatttgggaattgaaaagcttctagatcctgaagatgttgctgtgcatctccctgacaagaaatccataattatgtatttaacgtct
ctgtttgaggtgcttcctcagcaagtcacgatagatgccatccgagaggtggagactctcccaaggaagtataagaaagaatgtgaagaggaagaaattcatatc
cagagtgcagtgctggcagaggaaggccagagtccccgagctgagacccctagcaccgtcactgaagtggacatggatttggacagctaccagatagcgctagag
gaagtgctgacgtggctgctgtccgcggaggacacgttccaggagcaagatgacatttctgatgatgtcgaagaagtcaaagagcagtttgctacccatgaaact
tttatgatggagctgacagcacaccagagcagcgtggggagcgtcctgcaggctggcaaccagctgatgacacaagggactctgtcagaggaggaggagtttgag
atccaggaacagatgaccttgctgaatgcaaggtgggaggcgctccgggtggagagcatggagaggcagtcccggctgcacgacgctctgatggagctgcagaag
aaacagctgcagcagctctcaagctggctggccctcacagaagagcgcattcagaagatggagagcctcccgctgggtgatgacctgccctccctgcagaagctg
cttcaagaacataaaagtttgcaaaatgaccttgaagctgaacaggtgaaggtaaattccttaactcacatggtggtgattgtggatgaaaacagtggggagagt
gccacagctcttctggaagatcagttacagaaactgggtgagcgctggacagctgtatgccgctggactgaagaacgttggaacaggttgcaagaaatcagtatt
ctgtggcaggaattattggaagagcagtgtctgttggaggcttggctcaccgaaaaggaagaggctttgaataaagttcaaaccagcaactttaaagaccagaag
gaactaagtgtcagtgtccggcgtctggctatattgaaggaagacatggaaatgaagaggcagactctggatcaactgagtgagattggccaggatgtgggccaa
ttactcagtaatcccaaggcatctaagaagatgaacagtgactctgaggagctaacacagagatgggattctctggttcagagactcgaagactcttctaaccag
gtgactcaggcggtagcgaagctcggcatgtcccagattccacagaaggacctattggagaccgttcatgtgagagaacaagggatggtgaagaagcccaagcag
gaactgcctcctcctcccccaccaaagaagagacagattcacgtggacttagagaaactccgagacctgcagggagctatggacgacctggacgcagacatgaag
gaggtggaggctgtgcggaatggctggaagcccgtgggagacctgcttatagactccctgcaggatcacatcgagaaaaccctggcgtttagagaagaaattgca
ccaatcaacttaaaagtaaaaacaatgaatgacctgtccagtcagctgtctccacttgacttgcatccatctctaaagatgtctcgccagctggatgaccttaat
atgcgatggaaacttctacaggtttccgtggacgatcgccttaagcagctccaggaagcccacagagattttgggcctcttctcaacactttctgtccacttcag
tccagctgccgtggcagagatccatttcacataataaagtgccctattacatcaaccatcaaacacagacaacctgttgggatcatcctaaaatgactgagctct
tccaatcccttgctgatctgaataatgtacgtttctctgcctaccgcacagcaatcaaaatcgaaggctgcaaaaagcattatgtctggatctcttagagctgaa
tacgacgaatgaagttttcaagcagcacaaactgaaccaaaatgatcagctcctgagtgtcccagacgtcatcaactgtctgaccaccacttacgatgggcttga
gcagctgcacaaggacttggtcaatgttccactctgcgtcgatatgtgtctcaactggctgctcaacgtatacgacacgggccggactggaaaaattcgggtaca
gagtctgaagattggattgatgtctctctccaaaggcctcttagaagagaaatacagatgtctctttaaggaggtggcagggccaacagagatgtgtgaccagcg
gcagcttggcctgctacttcacgatgccatccagatccctaggcagctgggggaagtagcagcctttgggggcagtaacattgagcccagtgtccgcagctgctt
ccagcagaataacaacaagccagaaatcagtgtgaaggagtttatagactggatgcatttggaaccccagtccatggtgtggttgccggttctgcatcgggtcgc
agctgctgagactgcaaaacatcaggccaaatgcaacatctgcaaagaatgcccgattgttgggttcagatacaggagcctaaagcattttaattatgatgtctg
ccagagttgcttcttttctggaagaacagcaaagggccacaagttacattacccgatggtagaatactgcataccgacaacatctggggaagatgtgagagattt
cactaaggtgctgaagaacaagttcaggtccaagaaatattagccaaacatcctcggcttggctacctgcctgtccagaccgtgctggaaggggacaacttagaa
actcctatcacgctcatcagtatgtggccagagcactatgacccctcccagtcccctcagctgtttcatgatgacacccactcaagaatagagcaatacgctaca
cgactggcccagatggaaaggacaaacgggtccttcctaactgatagcagctctacaacaggaagcgtggaggatgagcatgccctcatccagcagtactgccag
accctgggcggggagtcacctgtgagtcagccgcagagtccagctcagatcctgaagtccgtggagagggaagagcgtggggaactggagcggatcattgctgac
ttggaggaagagcaaagaaatctgcaggtggagtatgagcagctgaaggagcagcacctaagaaggggtctccctgtgggctcccctccagactccatcgtatct
cctcaccacacatctgaggactcagaacttatagcagaagctaaactcctgcggcagcacaaagggcggctggaggcgaggatgcaaattttggaagatcacaat
aaacagctggagtctcagctgcaccgcctcagacagctcctggagcagcctgactctgactcccgcatcaatggtgtctccccctgggcttccccacagcattct
gcattgagctactcacttgacactgacccaggcccacagttccaccaggcagcatctgaggacctgctggccccacctcacgacactagcacggacctcacggac
gtgatggagcagatcaacagcacgtttccctcttgcagctcaaatgtccccagcaggccacaggcaatgtga
SEQ ID NO: 7
(TAT-HA Utrophin ΔR4-21)
MGYGRKKRRQRRRGGSTMSGYPYDVPDYAGSM [symbol] GDLEARPDDGQNEFSDIIKSRSDEHNDVQKK
TFTKWINARFSKSGKPPISDMFSDLKDGRKLLDLLEGLTGTSLPKERGSTRVHALNNVNRVLQVLHQNN
VDLVNIGGTDIVDGNPKLTLGLLWSIILHWQVKDVMKDIMSDLQQTNSEKILLSWVRQTTRPYSQVNV
LNFTTSWTDGLAFNAVLHRHKPDLFSWDRVVKMSPIERLEHAFSKAHTYLGIEKLLDPEDVAVHLPDK
KSIIMYLTSLFEVLPQQVTIDAIREVETLPRKYKKECEEEELHIQSAVLAEEGQSPRAETPSTVTEVDMDL
DSYQIALEEVLTWLLSAEDTFQEQDDISDDVEEVKEQFATHETFMMELTAHQSSVGSVLQAGNQLMTQ
GTLSEEEEFEIQEQMTLLNARWEALRVESMERQSRLHDALMELQKKQLQQLSSWLALTEERIQKMESL
PLGDDLPSLQKLLQEHKSLQNDLEAEQVKVNSLTHMVVIVDENSGESATALLEDQLQKLGERWTAVCR
WTEERWNRLQEISILWQELLEEQCLLEAWLTEKEEALNKVQTSNFKDQKELSVSVRRLAILKEDMEMK
RQTLDQLSEIGQDVGQLLSNPKASKKMNSDSEELTQRWDSLVQRLEDSSNQVTQAVAKLGMSQIPQKD
LLETVHVREQGMVKKPKQELPPPPPPKKRQIHVDLEKLRDLQGAMDDLDADMKEVEAVRNGWKPVG
DLLIDSLQDHIEKTLAFREEIAPINLKVKTMNDLSSQLSPLDLHPSLKMSRQLDDLNMRWKLLQVSVDD
RLKQLQEAHRDFGPSSQHFLSTSVQLPWQRSISHNKVPYYINHQTQTTCWDHPKMTELFQSLADLNNV
RFSAYRTAIKIRRLQKALCLDLLELNTTNEVFKQHKLNQNDQLLSVPDVINCLTTTYDGLEQLHKDLVN
VPLCVDMCLNWLLNVYDTGRTGKIRVQSLKIGLMSLSKGLLEEKYRCLFKEVAGPTEMCDQRQLGLLL
HDAIQIPRQLGEVAAFGGSNIEPSVRSCFQQNNNKPEISVKEFIDWMHLEPQSMVWLPVLHRVAAAETA
KHQAKCNICKECPIVGFRYRSLKHFNYDVCQSCFFSGRTAKGHKLHYPMVEYCIPTTSGEDVRDFTKVL
KNKFRSKKYFAKHPRLGYLPVQTVLEGDNLETPITLISMWPEHYDPSQSPQLFHDDTHSRIEQYATRLA
QMERTNGSFLTDSSSTTGSVEDEHALIQQYCQTLGGESPVSQPQSPAQILKSVEREERGELERIIADLEEE
QRNLQVEYEQLKEQHLRRGLPVGSPPDSIVSPHHTSEDSELIAEAKLLRQHKGRLEARMQILEDHNKQL
ESQLHRLRQLLEQPDSDSRINGVSPWASPQHSALSYSLDTDPGPQFHQAASEDLLAPPHDTSTDLTDVM
EQINSTFPSCSSNVPSRPQAM
SEQ ID NO: 7 Notes:
TAT PTD (bold underlined)
HA tag (underlined)
First three amino acids of utrophin (bold underlined italics)
SEQ ID NO: 8
(TAT Utrophin ΔR4-21)
atgggctacggccgcaagaaacgccgccagcgccgccgcgccaagtatggggaccttgaagccaggcctgatgatgggcagaacgaattcagtgacatcatta
agtccagatctgatgaacacaatgatgtacagaagaaaacctttaccaaatggataaacgctcgattttccaagagtgggaaaccacccatcagtgatatgtt
ctcagacctcaaagatgggagaaagctcttggatcttctcgaaggcctcacaggaacatcattgccaaaggaacgtggttccacaagggtgcatgccttaaac
aatgtcaaccgagtgctacaggttttacatcagaacaatgtggacttggtgaatattggaggcacggacattgtggatggaaatcccaagctgactttagggt
tactctggagcatcattctgcactggcaggtgaaggatgtcatgaaagatatcatgtcagacctgcagcagacaaacagcgagaagatcctgctgagctgggt
gcggcagaccaccaggccctacagtcaagtcaacgtcctcaacttcaccaccagctggaccgatggactcgcgttcaacgccgtgctccaccggcacaaacca
gatctcttcagctgggacagagtggtcaaaatgtccccaattgagagacttgaacatgcttttagcaaggcccacacttatttgggaattgaaaagcttctag
atcctgaagatgttgctgtgcatctccctgacaagaaatccataattatgtatttaacgtctctgtttgaggtgcttcctcagcaagtcacgatagatgccat
ccgagaggtggagactctcccaaggaagtataagaaagaatgtgaagaggaagaaattcatatccagagtgcagtgctggcagaggaaggccagagtccccga
gctgagacccctagcaccgtcactgaagtggacatggatttggacagctaccagatagcgctagaggaagtgctgacgtggctgctgtccgcggaggacacgt
tccaggagcaagatgacatttctgatgatgtcgaagaagtcaaagagcagtttgctacccatgaaacttttatgatggagctgacagcacaccagagcagcgt
ggggagcgtcctgcaggctggcaacaagctgatgacacaagggactctgtcagaggaggaggagtttgagatccaggaacagatgaccttgctgaatgcaagg
tgggaggcgctccgggtggagagcatggagaggcagtcccggctgcacgacgctctgatggagctgcagaagaaacagctgcagcagctctcaagctggctgg
ccctcacagaagagcgcattcagaagatggagagcctcccgctgggtgatgacctgccctccctgcagaagctgcttcaagaacataaaagtttgcaaaatga
ccttgaagctgaacaggtgaaggtaaattccttaactcacatggtggtgattgtggatgaaaacagtggggagagtgccacagctcttctggaagatcagtta
cagaaactgggtgagcgctggacagctgtatgccgctggactgaagaacgttggaacaggttgcaagaaatcagtattctgtggcaggaattattggaagagc
agtgtctgttggaggcttggctcaccgaaaaggaagaggctttgaataaagttcaaaccagcaactttaaagaccagaaggaactaagtgtcagtgtccggcg
tctggctatattgaaggaagacatggaaatgaagaggcagactctggatcaactgagtgagattggccaggatgtgggccaattactcagtaatcccaaggca
tctaagaagatgaacagtgactctgaggagctaacacagagatgggattctctggttcagagactcgaagactcttctaaccaggtgactcaggcggtagcga
agctcggcatgtcccagattccacagaaggacctattggagaccgttcatgtgagagaacaagggatggtgaagaagcccaagcaggaactgcctcctcctcc
cccaccaaagaagagacagattcacgtggacttagagaaactccgagacctgcagggagctatggacgacctggacgcagacatgaaggaggtggaggctgtg
cggaatggctggaagcccgtgggagacctgcttatagactccctgcaggatcacatcgagaaaaccctggcgtttagagaagaaattgcaccaatcaacttaa
aagtaaaaacaatgaatgacctgtccagtcagctgtctccacttgacttgcatccatctctaaagatgtctcgccagctggatgaccttaatatgcgatggaa
acttctacaggtttccgtggacgatcgccttaagcagctccaggaagcccacagagattttgggccatcttctcaacactttctgtccacttcagtccagctg
ccgtggcagagatccatttcacataataaagtgccctattacatcaaccatcaaacacagacaacctgttgggatcatcctaaaatgactgagctcttccaat
cccttgctgatctgaataatgtacgtttctctgcctaccgcacagcaatcaaaattcgaaggctgcaaaaagcattatgtctggatctcttagagctgaatac
gacgaatgaagttttcaagcagcacaaactgaaccaaaatgatcagctcctgagtgtcccagacgtcatcaactgtctgaccaccacttacgatgggcttgag
cagctgcacaaggacttggtcaatgttccactctgcgtcgatatgtgtctcaactggctgctcaacgtatacgacacgggccggactggaaaaattcgggtac
agagtctgaagattggattgatgtctctctccaaaggcctcttagaagagaaatacagatgtctctttaaggaggtggcagggccaacagagatgtgtgacca
gcggcagcttggcctgctacttcacgatgccatccagatccctaggcagctgggggaagtagcagcctttgggggcagtaacattgagcccagtgtccgcagc
tgcttccagcagaataacaacaagccagaaatcagtgtgaaggagtttatagactggatgcatttggaaccccagtccatggtgtggttgccggttctgcatc
gggtcgcagctgctgagactgcaaaacatcaggccaaatgcaacatctgcaaagaatgcccgattgttgggttcagatacaggagcctaaagcattttaatta
tgatgtctgccagagttgcttcttttctggaagaacagcaaagggccacaagttacattacccgatggtagaatactgcataccgacaacatctggggaagat
gtgagagatttcactaaggtgctgaagaacaagttcaggtccaagaaatattttgccaaacatcctcggcttggctacctgcctgtccagaccgtgctggaag
gggacaacttagaaactcctatcacgctcatcagtatgtggccagagcactatgacccctcccagtcccctcagctgtttcatgatgacacccactcaagaat
agagcaatacgctacacgactggcccagatggaaaggacaaacgggtccttcctaactgatagcagctctacaacaggaagcgtggaggatgagcatgccctc
atccagcagtactgccagaccctgggcggggagtcacctgtgagtcagccgcagagtccagctcagatcctgaagtccgtggagagggaagagcgtggggaac
tggagcggatcattgctgacttggaggaagagcaaagaaatctgcaggtggagtatgagcagctgaaggagcagcacctaagaaggggtctccctgtgggctc
ccctccagactccatcgtatctcctcaccacacatctgaggactcagaacttatagcagaagctaaactcctgcggcagcacaaagggcggctggaggcgagg
atgcaaattttggaagatcacaataaacagctggagtctcagctgcaccgcctcagacagctcctggagcagcctgactctgactcccgcatcaatggtgtct
ccccctgggcttccccacagcattctgcattgagctactcacttgacactgacccaggcccacagttccaccaggcagcatctgaggacctgctggccccacc
tcacgacactagcacggacctcacggacgtgatggagcagatcaacagcacgtttccctcttgcagctcaaatgtccccagcaggccacaggcaatgtga
SEQ ID NO: 9
(TAT Utrophin ΔR4-21)
MGYGRKKRRQRRR [symbol] GDLEARPDDGQNEFSDIIKSRSDEHNDVQKKTFTKWINARFSKSGKPPISDM
FSDLKDGRKLLDLLEGLTGTSLPKERGSTRVHALNNVNRVLQVLHQNNVDLVNIGGTDIVDGNPKLTL
GLLWSIILHWQVKDVMKDIMSDLQQTNSEKILLSWVRQTTRPYSQVNVLNFTTSWTDGLAFNAVLHRH
KPDLFSWDRVVKMSPIERLEHAFSKAHTYLGIEKLLDPEDVAVHLPDKKSIIMYLTSLFEVLPQQVTIDAI
REVETLPRKYKKECEEEELHIQSAVLAEEGQSPRAETPSTVTEVDMDLDSYQIALEEVLTWLLSAEDTFQ
EQDDISDDVEEVKEQFATHETFMMELTAHQSSVGSVLQAGNQLMTQGTLSEEEEFEIQEQMTLLNARW
EALRVESMERQSRLHDALMELQKKQLQQLSSWLALTEERIQKMESLPLGDDLPSLQKLLQEHKSLQND
LEAEQVKVNSLTHMVVIVDENSGESATALLEDQLQKLGERWTAVCRWTEERWNRLQEISILWQELLEE
QCLLEAWLTEKEEALNKVQTSNFKDQKELSVSVRRLAILKEDMEMKRQTLDQLSEIGQDVGQLLSNPK
ASKKMNSDSEELTQRWDSLVQRLEDSSNQVTQAVAKLGMSQIPQKDLLETVHVREQGMVKKPKQELP
PPPPPKKRQIHVDLEKLRDLQGAMDDLDADMKEVEAVRNGWKPVGDLLIDSLQDHIEKTLAFREEIAPI
NLKVKTMNDLSSQLSPLDLHPSLKMSRQLDDLNMRWKLLQVSVDDRLKQLQEAHRDFGPSSQHFLSTS
VQLPWQRSISHNKVPYYINHQTQTTCWDHPKMTELFQSLADLNNVRFSAYRTAIKIRRLQKALCLDLLE
LNTTNEVFKQHKLNQNDQLLSVPDVINCLTTTYDGLEQLHKDLVNVPLCVDMCLNWLLNVYDTGRTG
KIRVQSLKIGLMSLSKGLLEEKYRCLFKEVAGPTEMCDQRQLGLLLHDAIQIPRQLGEVAAFGGSNIEPS
VRSCFQQNNNKPEISVKEFIDWMHLEPQSMVWLPVLHRVAAAETAKHQAKCNICKECPIVGFRYRSLK
HFNYDVCQSCFFSGRTAKGHKLHYPMVEYCIPTTSGEDVRDFTKVLKNKFRSKKYFAKHPRLGYLPVQ
TVLEGDNLETPITLISMWPEHYDPSQSPQLFHDDTHSRIEQYATRLAQMERTNGSFLTDSSSTTGSVEDE
HALIQQYCQTLGGESPVSQPQSPAQILKSVEREERGELERIIADLEEEQRNLQVEYEQLKEQHLRRGLPV
GSPPDSIVSPHHTSEDSELIAEAKLLRQHKGRLEARMQILEDHNKQLESQLHRLRQLLEQPDSDSRINGV
SPWASPQHSALSYSLDTDPGPQFHQAASEDLLAPPHDTSTDLTDVMEQINSTFPSCSSNVPSRPQAM
SEQ ID NO: 9 Notes:
First three amino acids of utrophin (bold underlined italics)
SEQ ID NO: 10
MYGRKKRRQRRRGGSTMSGYPYDVPDYAGS [symbol] YGEHEASPDNGQNEFSDIIKSRSDEHNDVQKKT
FTKWINARFSKSGKPPINDMFTDLKDGRKLLDLLEGLTGTSLPKERGSTRVHALNNVNRVLQVLHQNN
VELVNIGGTDIVDGNPKLTLGLLWSIILHWQVKDVMKDVMSDLQQTNSEKILLSWVRQTTRPYSQVNV
LNFTTSWTDGLAFNAVLHRHKPDLFSWDRVVKMSPIERLEHAFSKAHTYLGIEKLLDPEDVAVHLPDK
KSIIMYLTSLFEVLPQQVTIDAIREVETLPRKYKKECEEEAINIQSTAPEEEHESPRAETPSTVTEVDMDLD
SYQIALEEVLTWLLSAEDTFQEQDDISDDVEEVKEQFATHEAFMMELTAHQSSVGSVLQAGNQLITQG
TLSDEEEFEIQEQMTLLNARWEALRVESMDRQSRLHDVLMELQKKQLQQLSAWLTLTEERIQKMETCP
LDDDVKSLQKLLEEHKSLQNDLEAEQVKVNSLTHMVVIVDENSGESATAILEDQLQKLGERWTAVCR
WTEERWNRLQEINILWQELLEEQCLLKAWLTEKEEALNKVQTSNFKDQKELSVSVRRLAILKEDMEMK
RQTLDQLSEIGQDVGQLLDNPKASKKINSDSEELTQRWDSLVQRLEDSSNQVTQAVAKLGMSQIPQKD
LLETVRVREQAITKKSKQELPPPPPPKKRQIHVDLEKLRDLQGAMDDLDADMKEAESVRNGWKPVGDL
LIDSLQDHIEKIMAFREEIAPINFKVKTVNDLSSQLSPLDLHPSLKMSRQLDDLNMRWKLLQVSVDDRLK
QLQEAHRDFGPSSQHFLSTSVQLPWQRSISHNKVPYYINHQTQTTCWDHPKMTELFQSLADLNNVRFSA
YRTAIKIRRLQKALCLDLLELNTTNEIFKQHKLNQNDQLLSVPDVINCLTTTYDGLEQMHKDLVNVPLC
VDMCLNWLLNVYDTGRTGKIRVQSLKIGLMSLSKGLLEEKYRYLFKEVAGPTEMCDQRQLGLLLHDAI
QIPRQLGEVAAFGGSNIEPSVRSCFQQNNNKPE
ISVKEFIDWMHLEPQSMVWLPVLHRVAAAETAKHQAKCNICKECPIVGFRYRSLKHFNYDVCQSCFFS
GRTAKGHKLHYPMVEYCIPTTSGEDVRDFTKVLKNKFRSKKYFAKHPRLGYLPVQTVLEGDNLETPITL
ISMWPEHYDPSQSPQLFHDDTHSRIEQYATRLAQMERTNGSFLTDSSSTTGSVEDEHALIQQYCQTLGG
ESPVSQPQSPAQILKSVEREERGELERIIADLEEEQRNLQVEYEQLKDQHLRRGLPVGSPPESIISPHHTSE
DSELIAEAKLLRQHKGRLEARMQILEDHNKQLESQLHRLRQLLEQPESDSRINGVSPWASPQHSALSYSL
DPDASGPQFHQAAGEDLLAPPHDTSTDLTEVMEQIHSTFPSCCPNVPSRPQAM
SEQ ID NO: 10 Notes:
TAT PTD (bold underlined)
HA tag (underlined)
First three amino acids of utrophin (bold underlined italics)
SEQ ID NO: 11
MYGRKKRRQRRRGGSTMSGYPYDVPDYAGS [symbol] YGEHEASPDNGQNEFSDIIKSRSDEHNDVQKKT
FTKWINARFSKSGKPPINDMFTDLKDGRKLLDLLEGLTGTSLPKERGSTRVHALNNVNRVLQVLHQNN
VELVNIGGTDIVDGNPKLTLGLLWSIILHWQVKDVMKDVMSDLQQTNSEKILLSWVRQTTRPYSQVNV
LNFTTSWTDGLAFNAVLHRHKPDLFSWDRVVKMSPIERLEHAFSKAHTYLGIEKLLDPEDVAVHLPDK
KSIIMYLTSLFEVLPQQVTIDAIREVETLPRKYKKECEEEAINIQSTAPEEEHESPRAETPSTVTEVDMDLD
SYQIALEEVLTWLLSAEDTFQEQDDISDDVEEVKEQFATHEAFMMELTAHQSSVGSVLQAGNQLITQG
TLSDEEEFEIQEQMTLLNARWEALRVESMDRQSRLHDVLMELQKKQLQQLSAWLTLTEERIQKMETCP
LDDDVKSLQKLLEEHKSLQNDLEAEQVKVNSLTHMVVIVDENSGESATAILEDQLQKLGERWTAVCR
WTEERWNRLQEINILWQELLEEQCLLKAWLTEKEEALNKVQTSNFKDQKELSVSVRRLAILKEDMEMK
RQTLDQLSEIGQDVGQLLDNPKASKKINSDSEELTQRWDSLVQRLEDSSNQVTQAVAKLGMSQIPQKD
LLETVRVREQAITKKSKQELPPPPPPKKRQIHVDLEKLRDLQGAMDDLDADMKEAESVRNGWKPVGDL
LIDSLQDHIEKIMAFREEIAPINFKVKTVNDLSSQLSPLDLHPSLKMSRQLDDLNMRWKLLQVSVDDRLK
QLQEAHRDFGPSSQHFLSTSVQLPWQRSISHNKVPYYINHQTQTTCWDHPKMTELFQSLADLNNVRFSA
YRTAIKIRRLQKALCLDLLELNTTNEIFKQHKLNQNDQLLSVPDVINCLTTTYDGLEQMHKDLVNVPLC
VDMCLNWLLNVYDTGRTGKIRVQSLKIGLMSLSKGLLEEKYRYLFKEVAGPTEMCDQRQLGLLLHDAI
QIPRQLGEVAAFGGSNIEPSVRSCFQQNNNKPEISVKEFIDWMHLEPQSMVWLPVLHRVAAAETAKHQ
AKCNICKECPIVGFRYRSLKHFNYDVCQSCFFSGRTAKGHKLHYPMVEYCIPTTSGEDVRDFTKVLKNK
FRSKKYFAKHPRLGYLPVQTVLEGDNLETPITLISMWPEHYDPSQSPQLFHDDTHSRIEQYATRLAQME
RTNGSFLTDSSSTTGSVEDEHALIQQYCQTLGGESPVSQPQSPAQILKSVEREERGELERIIADLEEEQRN
LQVEYEQLKDQHLRRGLPVGSPPESIISPHHTSEDSELIAEAKLLRQHKGRLEARMQILEDHNKQLESQL
HRLRQLLEQPESDSRINGVSPWASPQHSALSYSLDPDASGPQFHQAAGEDLLAPPHDTSTDLTEVMEQI
HSTFPSCCPNVPSRPQAM
SEQ ID NO: 11 Notes:
TAT PTD (bold underlined)
First three amino acids of utrophin (bold underlined italics)

1. An isolated polypeptide comprising:
a μ-utrophin region or an anti-dystrophinopathic fragment thereof operationally linked to a second region effective to transduce the fusion protein into mammalian muscle cells;
with the proviso that the isolated polypeptide does not include SEQ ID NO:1.

2. The isolated polypeptide of claim 1 wherein the μ-utrophin region or an anti-dystrophinopathic fragment thereof comprises a deletion of at least one spectrin-like repeat compared to native utrophin.

3. The isolated polypeptide of claim 1 wherein the second region comprises amino acids 3-13 of SEQ ID NO:7.

4. The isolated polypeptide of claim 1 wherein the second region comprises amino acids 21-29 of SEQ ID NO:7.

5. A composition comprising:
the isolated polypeptide of claim 1 or a pharmaceutically suitable salt thereof in combination with a pharmaceutically acceptable carrier.

6. An isolated nucleic acid expression construct encoding a polypeptide, the nucleic acid expression construct comprising:
a first nucleic acid region that encodes a μ-utrophin polypeptide or an anti-dystrophinopathic fragment thereof;
a second nucleic acid region that encodes an amino acid sequence effective to transduce the μ-utrophin polypeptide into mammalian muscle cells operationally linked to the first nucleic acid region;
with the proviso that the polypeptide does not include SEQ ID NO:1.

7. The isolated polynucleotide of claim 6 wherein the μ-utrophin polypeptide or an anti-dystrophinopathic fragment thereof comprises a deletion of at least one spectrin-like repeat compared to native utrophin.

8. The isolated polynucleotide of claim 6 wherein the second nucleic acid region encodes a polypeptide that comprises amino acids 3-13 of SEQ ID NO:7.

9. The isolated polynucleotide of claim 6 wherein the second nucleic acid region encodes a polypeptide that comprises amino acids 21-29 of SEQ ID NO:7.

10. A method of treating a dystrophinopathy in a subject, the method comprising:
administering to a subject in need such treatment an anti-dystrophinopathic amount of an isolated polypeptide comprising:
a μ-utrophin region or an anti-dystrophinopathic fragment thereof operationally linked to a second region effective to transduce the fusion protein into mammalian muscle cells;
with the proviso that the isolated polypeptide does not include SEQ ID NO:1.

11. The method of claim 10 wherein the dystrophinopathy comprises Duchenne muscular dystrophy.

12. The method of claim 10 wherein the isolated polypeptide is administered at least twice per week.

13. The method of claim 10 wherein the isolated polypeptide is administered for at least 13 weeks.

14. A method of isolating a polypeptide, the method comprising:
receiving a sample comprising a polypeptide comprising:
a μ-utrophin region or an anti-dystrophinopathic fragment thereof operationally linked to a second region effective to transduce the fusion protein into mammalian muscle cells; with the proviso that the isolated polypeptide does not include SEQ ID NO:1;
performing cation exchange chromatography on at least a portion of the sample; and
recovering the polypeptide at a purity of at least 86%.

15. The method of claim 14 wherein the polypeptide is recovered with a yield of at least 90%.

16. A method of isolating a polypeptide, the method comprising:
receiving a sample comprising a polypeptide comprising:
a μ-utrophin region or an anti-dystrophinopathic fragment thereof operationally linked to a second region effective to transduce the fusion protein into mammalian muscle cells; with the proviso that the isolated polypeptide does not include SEQ ID NO:1;
performing cation exchange chromatography on at least a portion of the sample; and
recovering the polypeptide at a yield of at least 90%.

17. A method of isolating a polypeptide that comprises a net negative charge, the method comprising:
obtaining a sample comprising a fusion polypeptide comprising:
the polypeptide comprising a net negative charge, and
a positively charged tag comprising at least 12 amino acids,
wherein at least one of the following is true:
the positively charged tag comprises at least one non-arginine amino acid residue, or
the positively charged tag is located at the N-terminal of the fusion polypeptide; and
performing cation exchange chromatography on at least a portion of the sample.
